(19) United States
(12) Patent Application Publication
(10) Pub. No.: US 2026/0070889 A1
(43) Pub. Date: Mar. 12, 2026

(54) 5-PYRIDINE-1H-INDAZOLE COMPOUND, PHARMACEUTICAL COMPOSITION, AND USE

(71) Applicant: CHINA PHARMACEUTICAL UNIVERSITY, Jiangsu (CN)

(72) Inventors: Zhiyu LI, Jiangsu (CN); Jinlei BIAN, Jiangsu (CN); Tianxing HU, Jiangsu (CN); Mai LIU, Jiangsu (CN); Jiaxing SONG, Jiangsu (CN); Zhixia QIU, Jiangsu (CN); Hongqian WU, Jiangsu (CN); Xi XU, Jiangsu (CN); Jubo WANG, Jiangsu (CN)

(73) Assignee: CHINA PHARMACEUTICAL UNIVERSITY, Jiangsu (CN)

(21) Appl. No.: 18/864,008

(22) PCT Filed: Nov. 22, 2022
(86) PCT No.: PCT/CN2022/133423

§ 371 Date: Nov. 8, 2024

(30) Foreign Application Priority Data
Aug. 24, 2022 (CN) 202211018173.7

(51) Int. Cl. C07D 401/14; A61K 31/517; A61K 31/519; A61P 19/02; C07D 413/14; C07D 471/04; C07D 491/048; C07D 495/04

(52) U.S. Cl. CPC C07D 401/14; A61K 31/517; A61K 31/519; A61P 19/02; C07D 413/14; C07D 471/04; C07D 491/048; C07D 495/04

(57) ABSTRACT

Disclosed in the present invention are a 5-pyridine-1H-indazole compound, a pharmaceutical composition, and a use. The structure of the compound is represented by formula I or II, and also comprised are an isomer or pharmaceutically acceptable salt thereof, or a mixture of said isomer and salt. The compound and the pharmaceutical composition thereof can effectively inhibit the activity of CLK2 and DYRK1A proteins, can be used for preparing drugs for treating osteoarthritis, can show the efficacy at a molecular level, has a more excellent therapeutic effect, and optimally, can reach a nano-molar concentration level. In addition, a preparation method for the compound is simple, convenient and easy to operate.

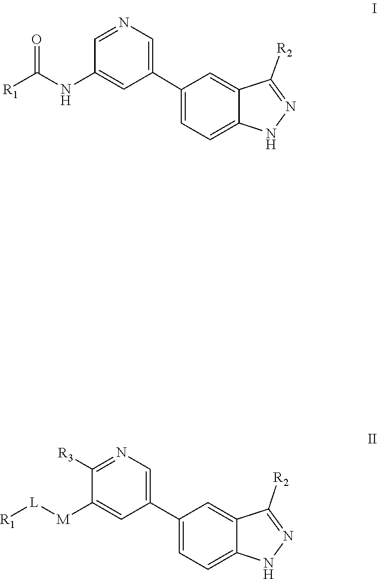

### TECHNICAL FIELD

The present invention relates to a 5-pyridine-1H-indazole compound, a pharmaceutical composition, and use, particularly to a 5-pyridine-1H-indazole compound which can be prepared to effectively inhibit activity of CLK2 or DYRK1A protein, a pharmaceutical composition, and use.

### DESCRIPTION OF RELATED ART

Osteoarthritis (OA) is characterized by synovial inflammation, cartilage loss, and subchondral bone remodeling. Despite the abundance of stem cells in the synovium of OA patients, the inability to regenerate joint cartilage is not due to a lack of stem cells. Instead, it is caused by improper differentiation of stem cells. The Wnt pathway plays a crucial role in organ development, cell differentiation, and tissue remodeling. Both abnormal activation and inhibition of the Wnt signaling pathway can lead to the disease. Therefore, the Wnt signaling pathway is a potential target for the treatment of osteoarthritis.

The Wnt signaling pathway is a group of multiple downstream channel signaling pathways triggered by the binding of the ligand protein Wnt to membrane protein receptors. Via this pathway, extracellular signals are transmitted into cells through an intracellular activation process of cell surface receptors. In the canonical Wnt pathway, when there is no Wnt protein on the surface of the cell membrane, the downstream β-catenin protein will be degraded by the glycogen synthase kinase 3 (GSK3) complex in the cytoplasm, preventing it from entering the nucleus to start the transcription of related Wnt genes; when there is Wnt protein on the surface of the cell membrane, it can inhibit the GSK3 complex, thereby allowing the β-catenin protein to accumulate in the nucleus and finally start the transcription of genes related to the Wnt pathway. There is a delicate balance between joint homeostasis and the Wnt pathway, and disruption of this balance may lead to OA.

The protein kinase family CLK (CDK-likekinase) is a dual specificity protein kinase that can regulate intracellular signaling through phosphorylation of tyrosine, serine, or threonine residue substrate proteins. It has four subtypes (CLK1, CLK2, CLK3, and CLK4), and the C segments of the proteins encoded by the four subtypes all have highly conserved gene sequences and have structurally similar amino acid sequences. The CLK2 subtype is present in most eukaryotes, and it participates in the phosphorylation of SR (serine/arginine) protein domains, thereby regulating the selective cleavage of RNA, plays an important role in gluconeogenesis and fatty acid oxidation in the liver, and is also a therapeutic target for liver cancer, breast cancer, and Alzheimer's disease and a potential therapeutic target for Wnt pathway and osteoarthritis.

Dual specificity tyrosine phosphorylation regulated kinase 1A (DYRK1A) belongs to the DYRK family and is highly conserved evolutionarily. In mammals, the DYRK family has five different subtypes, and only DYRK1A is located in the DSCR region of human chromosome 21. DYRK1A is expressed by the dyrk1a gene, and the encoded mature protein consists of 763 amino acids, including a protein kinase domain and other special structures. Many important proteins can serve as substrates for DYRK1A and are regulated by it, thus participating in various biological functions in cells. For example, neural development, cell proliferation and differentiation, tumorigenesis, and neurodegenerative diseases.

At present, no CLK2 inhibitor drug for treating osteoarthritis is successfully marketed, and only one small molecule drug SM-04690 enters clinical stage III, so that the clinical requirement cannot be met. SM04690 has significant CLK2 inhibitory activity but is not selective to the CLK family, which may have some side effects; in addition, it has insufficient inhibitory activity on the DYRK1A target and poor water solubility, so the druggability needs to be improved.

### SUMMARY OF INVENTION

Objective: Aiming at the problems of insufficient selectivity for the CLK family, insufficient inhibitory activity on the DYRK1A target, and the like, of the existing compounds, the present invention aims to provide a 5-pyridine-1H-indazole compound which can specifically inhibit activity of CLK2 and DYRK1A proteins, a pharmaceutical composition, and use.

Technical scheme: As a first aspect to which the present invention relates, the 5-pyridine-1H-indazole compound of the present invention has a structure of formula I or II, and further comprises an isomer thereof, a pharmaceutically acceptable salt thereof, or a mixture thereof:
R₁ is selected from the following groups substituted with one or more of hydrogen, halogen, methoxy, trifluoromethyl, nitro, hydroxy, amino, azido, sulfonic acid group, or 3-6 membered ring: hydrogen, linear or branched C₁-C₁₀ alkyl, phenyl, a 4-6 membered heterocyclic ring, or a 4-6 membered ring;
L and M are selected from —CH₂—, —NH—, —O—, or a chemical bond;
R₂ is selected from the following groups:

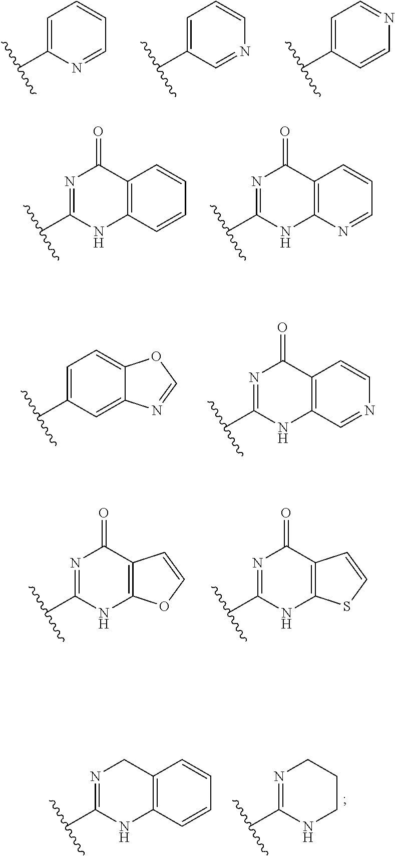

R₃ is selected from hydrogen or C₁-C₄ alkyl.

The present invention synthesizes a series of derivatives through reasonable drug design. Biological activity evaluation shows that the designed compound has significant inhibitory activity against CLK2, good selectivity for CLK family members, and significant inhibitory activity against DYRK1A.

Preferably, in the structure of the compound described above:
R₁ is selected from isobutyl, cyclopropylmethyl, cyclopentyl, α-aminoisopentyl, 3,3-difluorotetrahydropyrrolyl, hydrogen, morpholinyl, methyl, tert-butyl, ethanesulfonyl, or hydroxy;
R₂ is selected from the following groups:

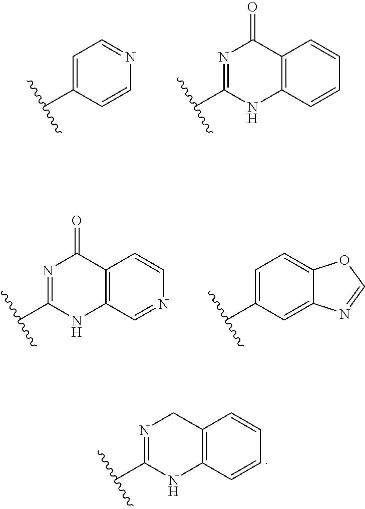

More preferably, the compound described above is selected from any one of the following compounds:

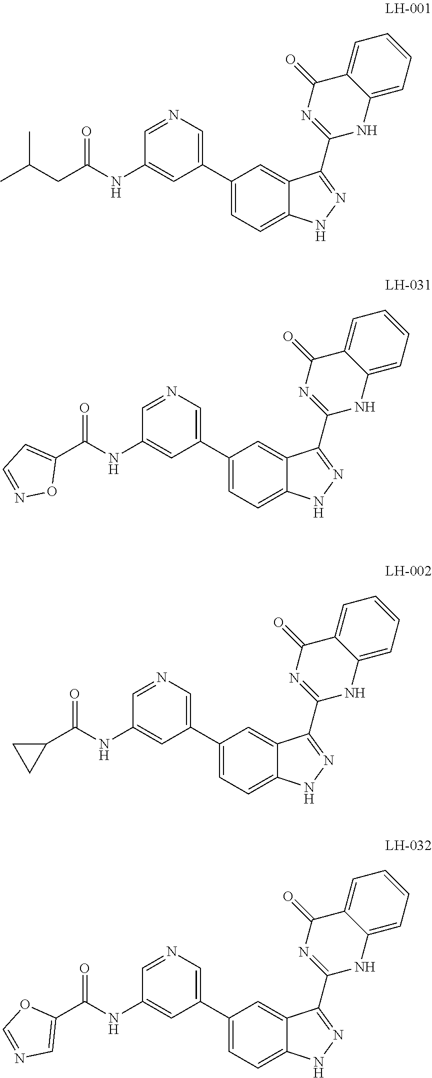 [SMILES: CC(C)CC(=O)Nc1cncc(-c2ccc3[nH]nc(-c4nc(=O)c5ccccc5[nH]4)c3c2)c1 ; O=C(Nc1cncc(-c2ccc3[nH]nc(-c4nc(=O)c5ccccc5[nH]4)c3c2)c1)c1ccno1 ; O=C(Nc1cncc(-c2ccc3[nH]nc(-c4nc(=O)c5ccccc5[nH]4)c3c2)c1)C1CC1 ; O=C(Nc1cncc(-c2ccc3[nH]nc(-c4nc(=O)c5ccccc5[nH]4)c3c2)c1)c1cnco1]

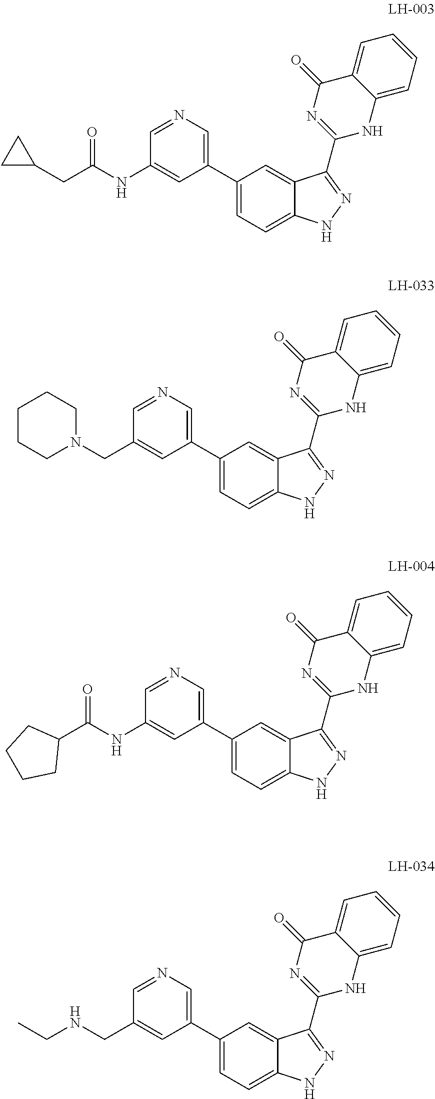 [SMILES: O=C(CC1CC1)Nc1cncc(-c2ccc3[nH]nc(-c4nc(=O)c5ccccc5[nH]4)c3c2)c1 ; O=c1nc(-c2n[nH]c3ccc(-c4cncc(CN5CCCCC5)c4)cc23)[nH]c2ccccc12 ; O=C(Nc1cncc(-c2ccc3[nH]nc(-c4nc(=O)c5ccccc5[nH]4)c3c2)c1)C1CCCC1 ; CCNCc1cncc(-c2ccc3[nH]nc(-c4nc(=O)c5ccccc5[nH]4)c3c2)c1]

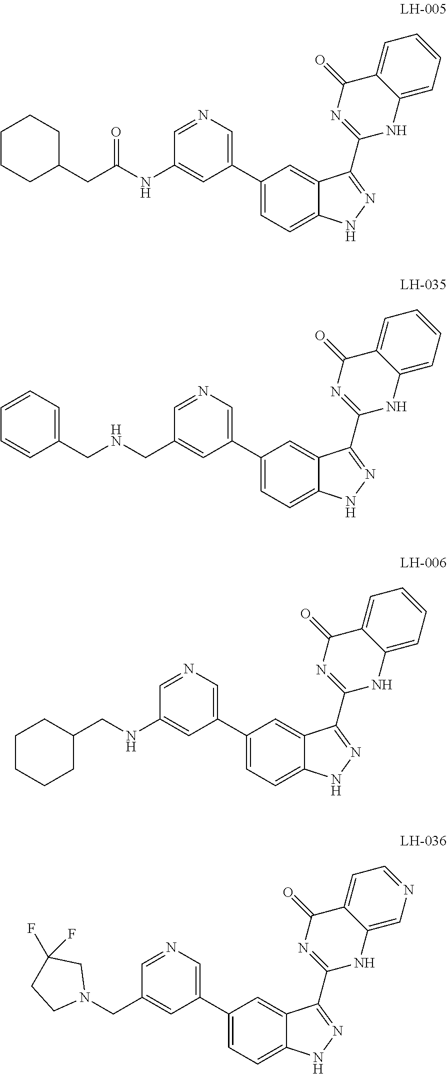 [SMILES: O=C(CC1CCCCC1)Nc1cncc(-c2ccc3[nH]nc(-c4nc(=O)c5ccccc5[nH]4)c3c2)c1 ; O=c1nc(-c2n[nH]c3ccc(-c4cncc(CNCc5ccccc5)c4)cc23)[nH]c2ccccc12 ; O=c1nc(-c2n[nH]c3ccc(-c4cncc(NCC5CCCCC5)c4)cc23)[nH]c2ccccc12 ; O=c1nc(-c2n[nH]c3ccc(-c4cncc(CN5CCC(F)(F)C5)c4)cc23)[nH]c2cnccc12]

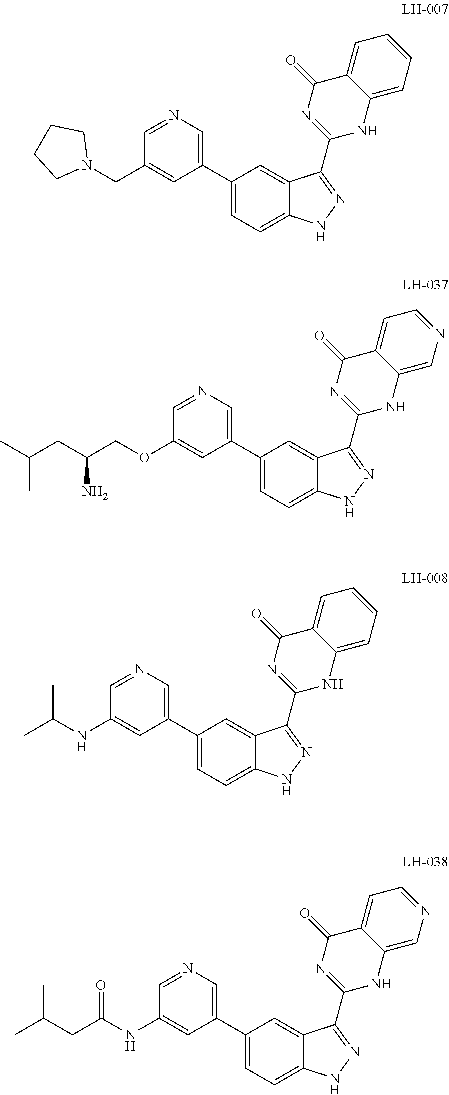 [SMILES: O=c1nc(-c2n[nH]c3ccc(-c4cncc(CN5CCCC5)c4)cc23)[nH]c2ccccc12 ; CC(C)C[C@H](N)COc1cncc(-c2ccc3[nH]nc(-c4nc(=O)c5ccncc5[nH]4)c3c2)c1 ; CC(C)Nc1cncc(-c2ccc3[nH]nc(-c4nc(=O)c5ccccc5[nH]4)c3c2)c1 ; CC(C)CC(=O)Nc1cncc(-c2ccc3[nH]nc(-c4nc(=O)c5ccncc5[nH]4)c3c2)c1]

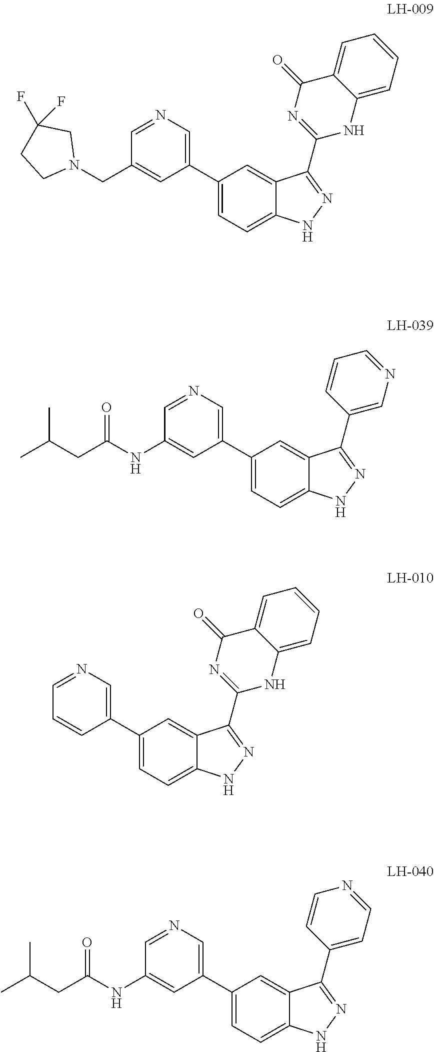 [SMILES: O=c1nc(-c2n[nH]c3ccc(-c4cncc(CN5CCC(F)(F)C5)c4)cc23)[nH]c2ccccc12 ; CC(C)CC(=O)Nc1cncc(-c2ccc3[nH]nc(-c4cccnc4)c3c2)c1 ; O=c1nc(-c2n[nH]c3ccc(-c4cccnc4)cc23)[nH]c2ccccc12 ; CC(C)CC(=O)Nc1cncc(-c2ccc3[nH]nc(-c4ccncc4)c3c2)c1]

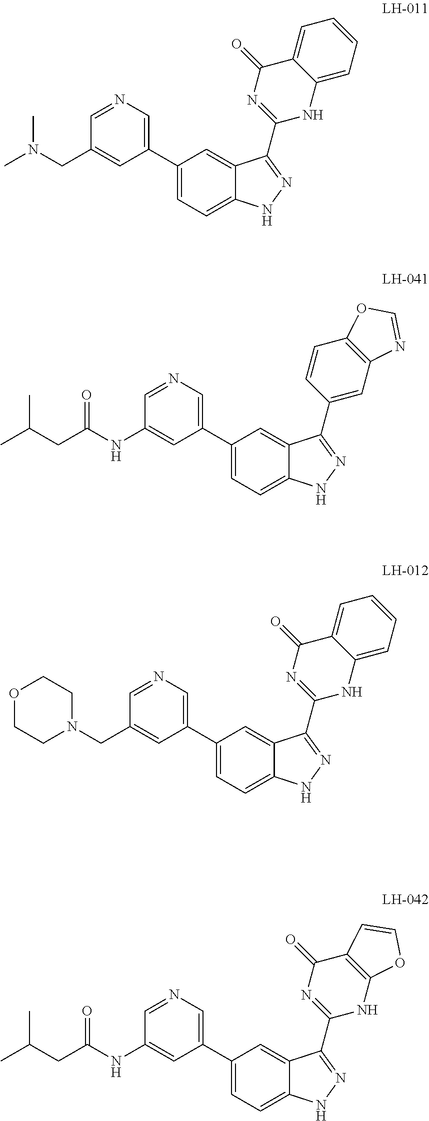 [SMILES: CN(C)Cc1cncc(-c2ccc3[nH]nc(-c4nc(=O)c5ccccc5[nH]4)c3c2)c1 ; CC(C)CC(=O)Nc1cncc(-c2ccc3[nH]nc(-c4ccc5ocnc5c4)c3c2)c1 ; O=c1nc(-c2n[nH]c3ccc(-c4cncc(CN5CCOCC5)c4)cc23)[nH]c2ccccc12 ; CC(C)CC(=O)Nc1cncc(-c2ccc3[nH]nc(-c4nc(=O)c5ccoc5[nH]4)c3c2)c1]

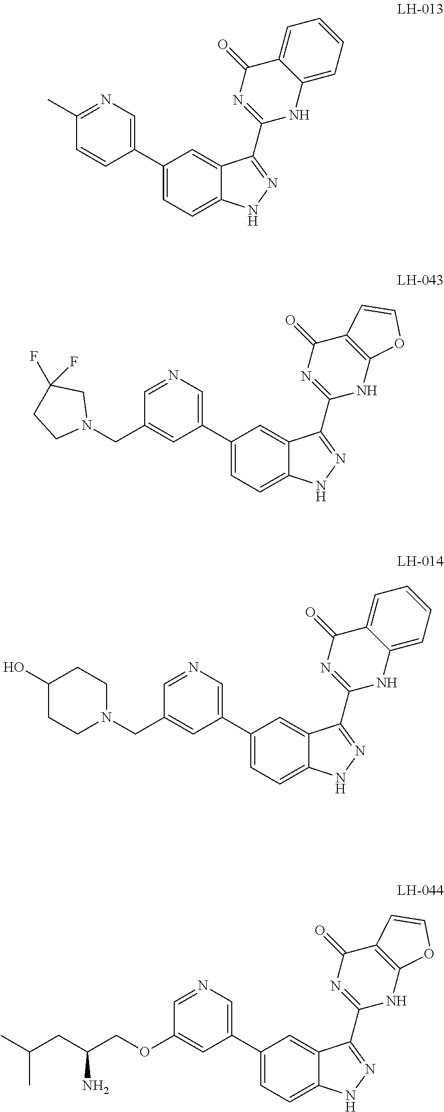 [SMILES: Cc1ccc(-c2ccc3[nH]nc(-c4nc(=O)c5ccccc5[nH]4)c3c2)cn1 ; O=c1nc(-c2n[nH]c3ccc(-c4cncc(CN5CCC(F)(F)C5)c4)cc23)[nH]c2occc12 ; O=c1nc(-c2n[nH]c3ccc(-c4cncc(CN5CCC(O)CC5)c4)cc23)[nH]c2ccccc12 ; CC(C)C[C@H](N)COc1cncc(-c2ccc3[nH]nc(-c4nc(=O)c5ccoc5[nH]4)c3c2)c1]

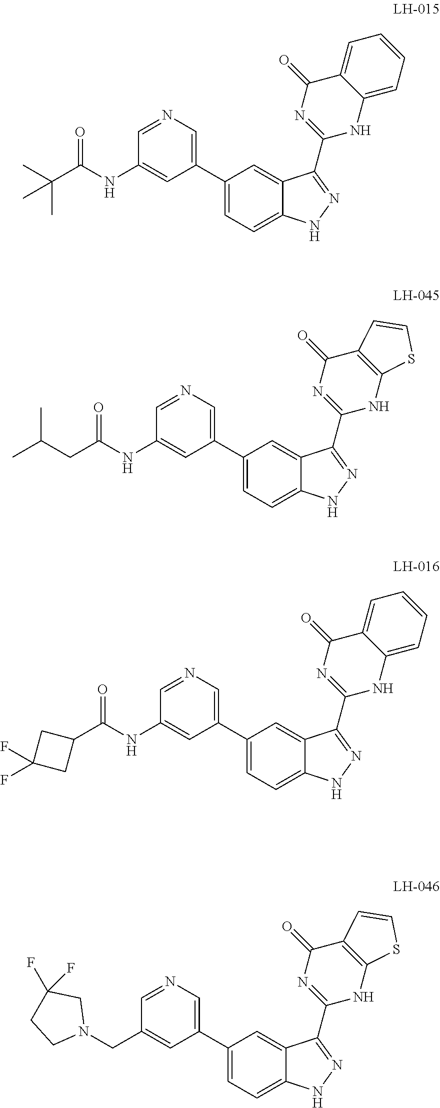 [SMILES: CC(C)(C)C(=O)Nc1cncc(-c2ccc3[nH]nc(-c4nc(=O)c5ccccc5[nH]4)c3c2)c1 ; CC(C)CC(=O)Nc1cncc(-c2ccc3[nH]nc(-c4nc(=O)c5ccsc5[nH]4)c3c2)c1 ; O=C(Nc1cncc(-c2ccc3[nH]nc(-c4nc(=O)c5ccccc5[nH]4)c3c2)c1)C1CC(F)(F)C1 ; O=c1nc(-c2n[nH]c3ccc(-c4cncc(CN5CCC(F)(F)C5)c4)cc23)[nH]c2sccc12]

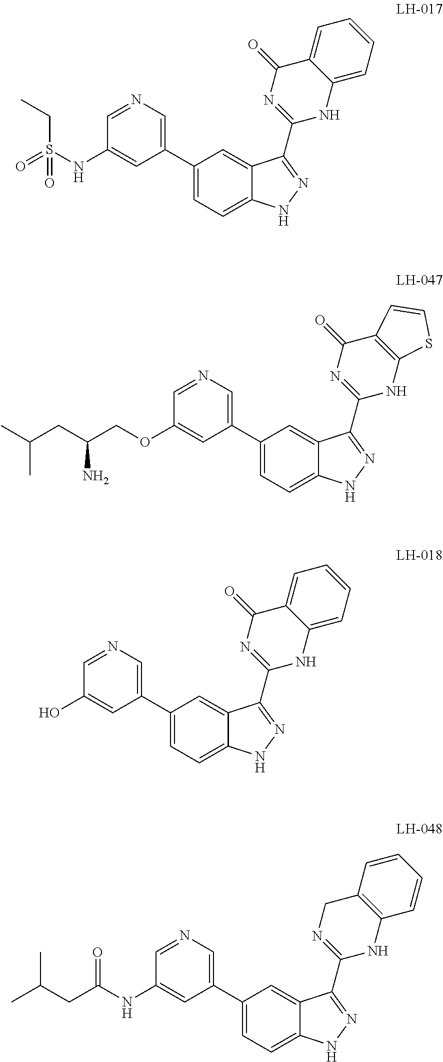 [SMILES: CCS(=O)(=O)Nc1cncc(-c2ccc3[nH]nc(-c4nc(=O)c5ccccc5[nH]4)c3c2)c1 ; CC(C)C[C@H](N)COc1cncc(-c2ccc3[nH]nc(-c4nc(=O)c5ccsc5[nH]4)c3c2)c1 ; O=c1nc(-c2n[nH]c3ccc(-c4cncc(O)c4)cc23)[nH]c2ccccc12 ; CC(C)CC(=O)Nc1cncc(-c2ccc3[nH]nc(C4=NCc5ccccc5N4)c3c2)c1]

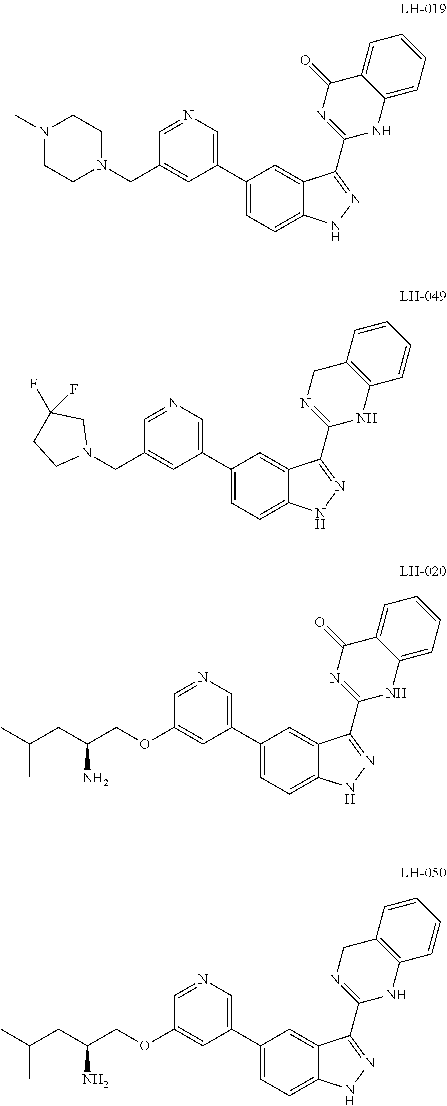 [SMILES: CN1CCN(Cc2cncc(-c3ccc4[nH]nc(-c5nc(=O)c6ccccc6[nH]5)c4c3)c2)CC1 ; FC1(F)CCN(Cc2cncc(-c3ccc4[nH]nc(C5=NCc6ccccc6N5)c4c3)c2)C1 ; CC(C)C[C@H](N)COc1cncc(-c2ccc3[nH]nc(-c4nc(=O)c5ccccc5[nH]4)c3c2)c1 ; CC(C)C[C@H](N)COc1cncc(-c2ccc3[nH]nc(C4=NCc5ccccc5N4)c3c2)c1]

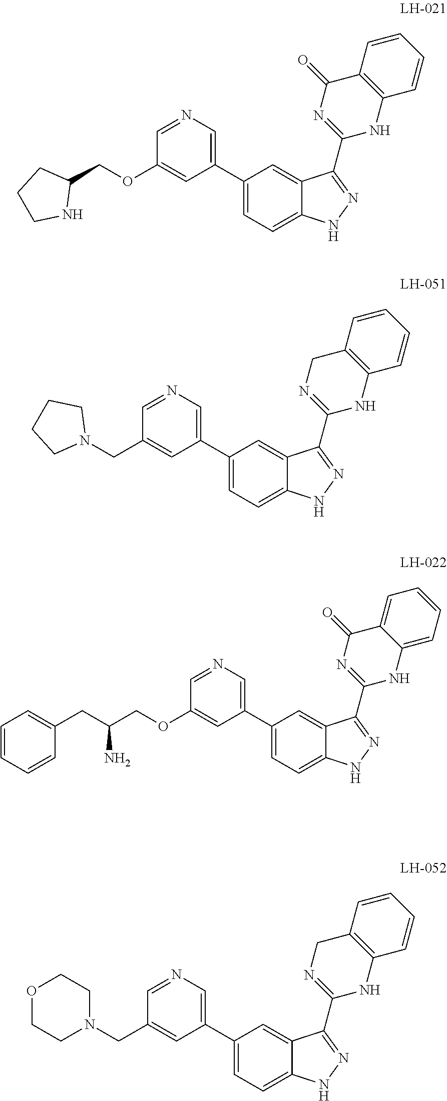

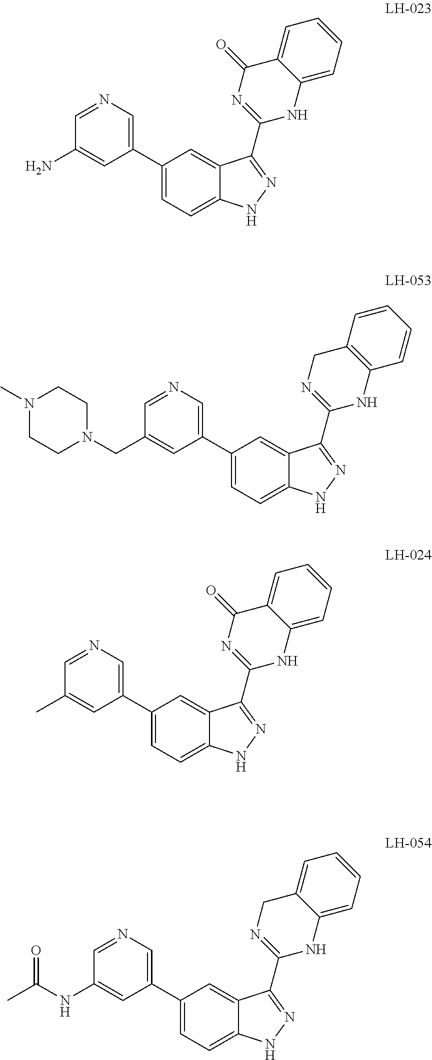 [SMILES: Nc1cncc(-c2ccc3[nH]nc(-c4nc(=O)c5ccccc5[nH]4)c3c2)c1 ; CN1CCN(Cc2cncc(-c3ccc4[nH]nc(C5=NCc6ccccc6N5)c4c3)c2)CC1 ; Cc1cncc(-c2ccc3[nH]nc(-c4nc(=O)c5ccccc5[nH]4)c3c2)c1 ; CC(=O)Nc1cncc(-c2ccc3[nH]nc(C4=NCc5ccccc5N4)c3c2)c1]

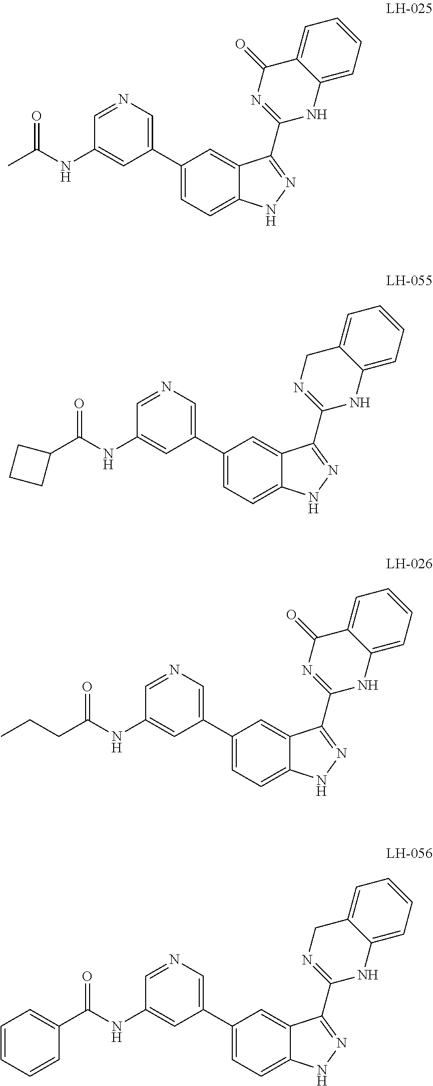 [SMILES: CC(=O)Nc1cncc(-c2ccc3[nH]nc(-c4nc(=O)c5ccccc5[nH]4)c3c2)c1 ; O=C(Nc1cncc(-c2ccc3[nH]nc(C4=NCc5ccccc5N4)c3c2)c1)C1CCC1 ; CCCC(=O)Nc1cncc(-c2ccc3[nH]nc(-c4nc(=O)c5ccccc5[nH]4)c3c2)c1 ; O=C(Nc1cncc(-c2ccc3[nH]nc(C4=NCc5ccccc5N4)c3c2)c1)c1ccccc1]

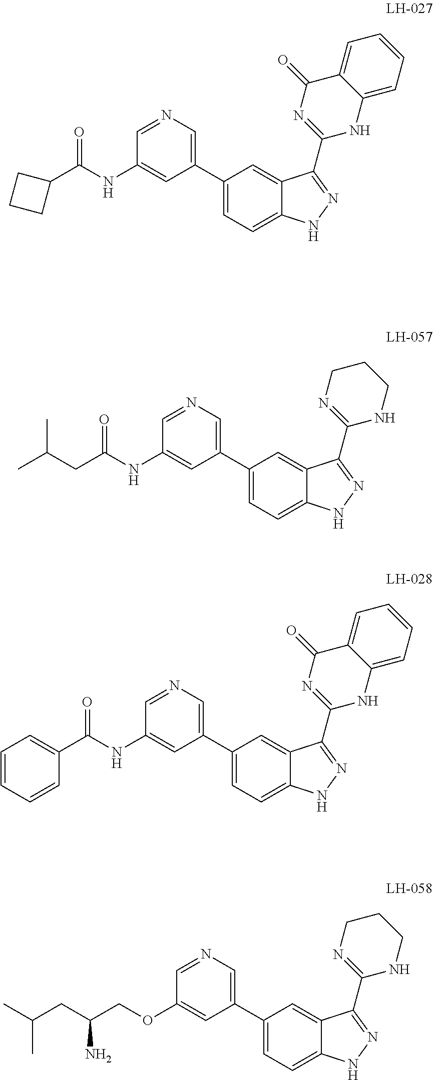 [SMILES: O=C(Nc1cncc(-c2ccc3[nH]nc(-c4nc(=O)c5ccccc5[nH]4)c3c2)c1)C1CCC1 ; CC(C)CC(=O)Nc1cncc(-c2ccc3[nH]nc(C4=NCCCN4)c3c2)c1 ; O=C(Nc1cncc(-c2ccc3[nH]nc(-c4nc(=O)c5ccccc5[nH]4)c3c2)c1)c1ccccc1 ; CC(C)C[C@H](N)COc1cncc(-c2ccc3[nH]nc(C4=NCCCN4)c3c2)c1]

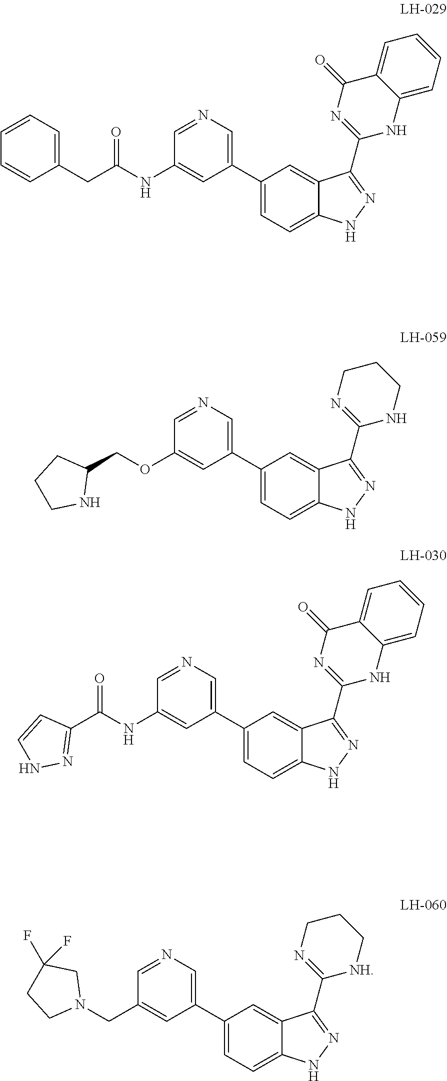

The pharmaceutically acceptable salt of the compound described above is a salt formed by the compound described above and the following acids: hydrochloric acid, sulfuric acid, phosphoric acid, carbonic acid, nitric acid, hydrobromic acid, hydroiodic acid, maleic acid, fumaric acid, tartaric acid, citric acid, malic acid, methanesulfonic acid, p-toluenesulfonic acid, naphthalenesulfonic acid, succinic acid, acetic acid, mandelic acid, isobutyric acid, or malonic acid.

As a second aspect to which the present invention relates, the compound described above and a pharmaceutically acceptable carrier form a pharmaceutical composition to prepare common pharmaceutical formulations, such as tablets, capsules, syrups, suspending agents, or injections, and common pharmaceutical adjuvants, such as perfuming agents, sweeteners, liquid/solid fillers, diluents, etc., can be added to the formulations.

In a third aspect to which the present invention relates, the compound described above or a pharmaceutical composition thereof can be prepared into a CLK2 protein inhibitor drug, and also can be prepared into a DYRK1A protein inhibitor drug, which is specifically used for treating inflammation, including osteoarthritis, tendinopathy, or rheumatoid arthritis, having a chondroprotective effect.

Beneficial Effects: The present invention has the following remarkable advantages compared with the prior art:
(1) such compounds and the pharmaceutical composition thereof can effectively inhibit activity of CLK2 protein and DYRK1A protein, and the IC₅₀ values of enzyme level inhibition are less than 100 nM, preferably less than 10 nM, and significantly down-regulate the expression level of the protease related to cartilage decomposition in an inflammation model animal, exerting a chondroprotective effect;
(2) such compounds and the pharmaceutical composition thereof are widely used and can be prepared into drugs for treating osteoarthritis; the drugs can exert drug effects at a molecular level and an animal level, and have more excellent treatment effect, which can optimally reach the nano-molar concentration level;
(3) the preparation method for the compounds is simple, convenient, and easy to operate.

### BRIEF DESCRIPTION OF DRAWINGS

FIG. 1 shows RT-qPCR results for cartilage of ACLT model rats at week 5 (*P<0.05, **P<0.01. ***P<0.001, ****P<0.0001).

### DESCRIPTION OF EMBODIMENTS

The technical scheme of the present invention will be further described below with reference to the examples.

### Example 1: Synthesis of LH-001

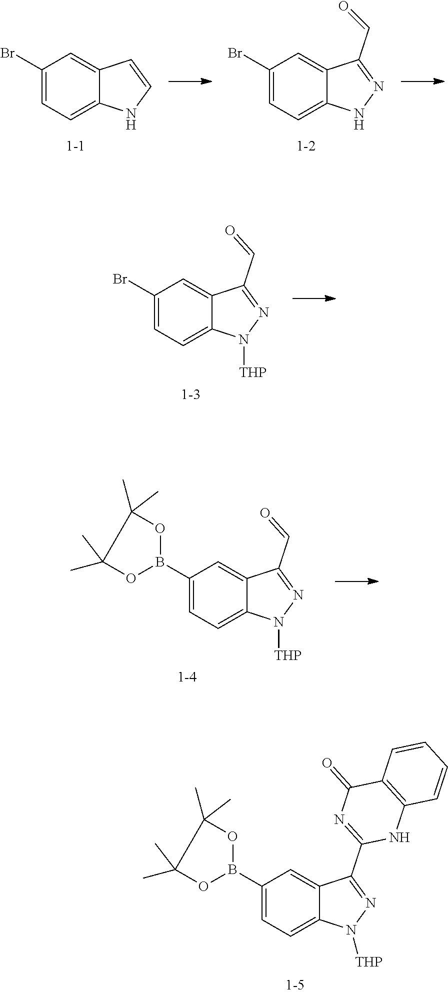

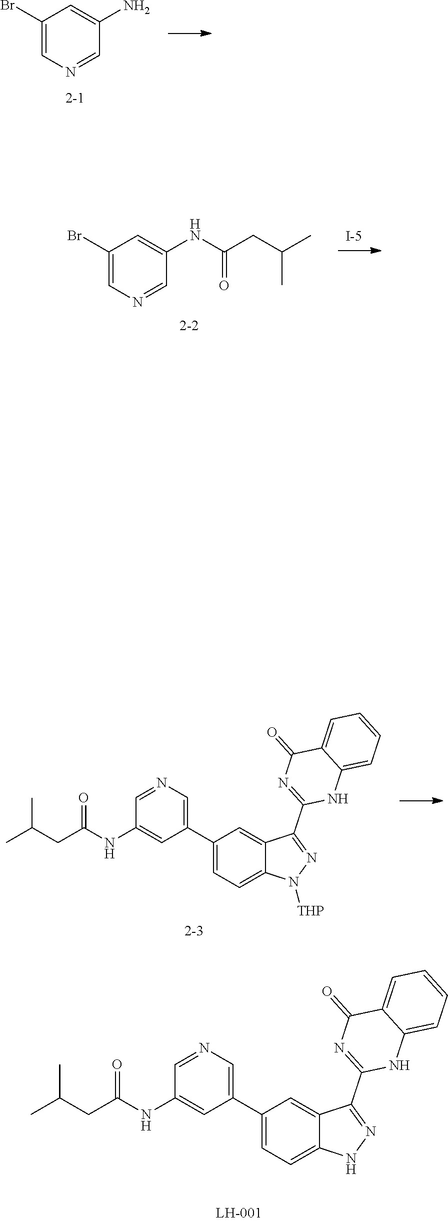

### Synthesis of Intermediate 1-2:

Starting material 1-1 (5-bromoindole, 0.1 mmol) and acetone were added into a three-necked flask. The mixture was cooled to 0° C. in an ice salt bath. An aqueous solution of sodium nitrite (0.8 mmol) was added dropwise, with the temperature maintained at no higher than 0° C. After the addition was completed, dilute hydrochloric acid (2 N HCl) was added dropwise. The mixture was then warmed to room temperature and reacted for 4 h. The mixture was concentrated by rotary evaporation and filtered under vacuum to give a crude product. The crude product was slurried with dichloromethane to give brownish red solid 1-2. ¹H NMR (300 MHz, DMSO-d₆) δ 12.29 (s, 1H), 9.80 (s, 1H), 7.88 (d, J=1.2 Hz, 1H). 7.44-7.31 (m, 2H) ppm. HR-MS(ESI): Calculated for C₈H₆BrN₂O [M+H]⁺: 224.9664. found 224.9657. The yield was 67%.

### Synthesis of Intermediate 1-3:

Intermediate 1-2 (0.1 mmol) was added to a three-necked flask. Tetrahydrofuran (80 mL) and p-toluenesulfonic acid (0.02 mmol) were added, and 3,4-dihydropyran (0.2 mmol) was added dropwise at room temperature. The mixture was then warmed to 65° C. and reacted for 8 h. After the reaction was completed as monitored by TLC, the reaction mixture was concentrated by rotary evaporation under reduced pressure to remove the solvent and purified by silica gel column chromatography to give a crude product of intermediate I-3. The crude product was slurried with petroleum ether to give white solid I-3. ¹H NMR (300 MHz, DMSO-d₆) δ 9.80 (s, 1H), 7.88 (s. 1H), 7.37 (d, J=1.0 Hz, 2H), 6.27 (t, J=6.9 Hz, 1H), 3.90 (dt, J=11.4, 7.0 Hz, 1H), 3.77 (dt, J=11.5, 7.0 Hz, 1H), 2.52-2.32 (m, 1H), 2.12-1.91 (m, 2H), 1.83-1.64 (m, 1H), 1.58-1.53 (m, 2H) ppm. HR-MS(ESI): Calculated for C₁₃H₁₄BrN₂O₂ [M+H]⁺: 309.0239. found 309.0239. The yield was 74%.

### Synthesis of Intermediate I-4:

Intermediate 1-3 (0.1 mmol) was added to a single-necked flask, and bis(pinacolato)diboron (0.12 mmol), potassium acetate (0.3 mmol), Pd(dppf)Cl₂ (0.005 mmol), and dioxane (26 mL) were added. The mixture was warmed at reflux for 6 h. After the reaction was completed as monitored by TLC, the reaction mixture was cooled, filtered under vacuum, concentrated by rotary evaporation, and purified by silica gel column chromatography to give white solid 1-4. ¹H NMR (300 MHz, DMSO-d₆) δ 9.99 (s, 1H), 8.07 (d, J=1.6 Hz, 1H), 7.46 (d, J=7.5 Hz, 1H), 7.27 (dd, J=7.5, 1.6 Hz, 1H), 6.25 (t, J=6.9 Hz, 1H), 3.90 (dt, J=11.4, 7.0 Hz, 1H), 3.77 (dt, J=11.4, 7.0 Hz, 1H), 2.52-2.32 (m, 1H), 2.12-1.91 (m, 2H), 1.86-1.48 (m, 3H), 1.41 (d, J=15.1 Hz, 12H) ppm. HR-MS(ESI): Calculated for C₁₉H₂₆BN₂O₄ [M+H]⁺: 357.1986. found 357.1983. The yield was 89%.

### Synthesis of Intermediate I-5:

Intermediate 1-4 (0.1 mmol) was added to a single-necked flask and dissolved with a mixed solvent of ethanol:dichloromethane (5:1). Anthranilamide (0.1 mmol) and iodine (0.1 mmol) were then added, and the mixture was refluxed at 80° C. for 30 min-60 min. After the TLC reaction was completed, 5% sodium thiosulfate was added to quench the reaction, and the mixture was extracted with dichloro and saturated brine. The organic phases were combined, dried, concentrated, and purified by column chromatography to give a crude product. The crude product was slurried with (petroleum ether:ethyl acetate=1:1) to give pure product I-5. ¹H NMR (300 MHz, DMSO-d₆) δ 12.35 (s, 1H), 8.13 (dd, J=7.6, 1.9 Hz, 2H), 7.66 (dd, J=7.5, 2.1 Hz, 1H), 7.56 (d, J=7.4 Hz, 1H), 7.50-7.32 (m, 2H), 7.13 (td, J=7.5, 2.0 Hz, 1H), 6.31 (t, J=6.9 Hz, 1H), 3.90 (dt, J=11.4, 7.0 Hz, 1H), 3.77 (dt, J=11.4, 7.0 Hz, 1H), 2.52-2.32 (m, 1H), 2.12-1.91 (m, 2H), 1.86-1.48 (m, 3H), 1.42 (d, J=15.1 Hz, 12H) ppm. HR-MS(ESI): Calculated for C₂₆H₃₀BN₄O₄ [M+H]⁺: 473.2360. found 473.2357. The yield was 68%.

### Synthesis of Intermediate 2-2:

Isovaleric acid (0.1 mmol) and HATU (0.3 mmol) were dissolved in 40 mL of tetrahydrofuran. DIPEA (0.3 mmol) was then added, and the mixture was stirred at room temperature for about 15 min. Starting material 2-1 (0.1 mmol) was then added, and the mixture was stirred at room temperature for 1-3 h. After starting material 2-1 was completely reacted as detected by TLC, the reaction was stopped, and the reaction mixture was separated and purified by column chromatography to give intermediate 2-2.

### Synthesis of Intermediate 2-3:

Intermediate 2-2 (0.1 mmol), intermediate 1-5 (0.1 mmol), sodium carbonate (0.3 mmol), Pd(dppf)Cl₂ (0.005 mmol), dioxane (9 mL), and water (1.5 mL) were added to a single-necked flask, and the system was purged of air with nitrogen 4 times. The mixture was warmed at reflux for 8 h. After the mixture was cooled, the mixture was filtered under vacuum, concentrated to dryness by rotary evaporation, and purified by silica gel column chromatography to give intermediate 2-3.

### Synthesis of LH-001:

Intermediate 2-3 (0.1 mmol) was dissolved in 5 mL of dichloromethane. Trifluoroacetic acid (1 mmol) and triethylsilane (0.1 mmol) were added at room temperature, and the mixture was reacted at room temperature for 16-24 h. After the reaction was completed as detected by TLC, the reaction mixture was concentrated under reduced pressure and extracted with dichloromethane and saturated sodium bicarbonate. The organic phases were combined, dried, and concentrated to give the final product LH-001. ¹H NMR (300 MHz, DMSO-d₆) δ 14.16 (s, 1H), 10.57 (s, 1H), 8.93 (t, J=12.4 Hz, 3H), 8.67 (d, J=2.1 Hz, 1H), 8.26-8.17 (m, 1H), 7.98-7.82 (m, 4H), 7.61-7.51 (m, 1H), 6.30 (s, 1H), 2.34 (d, J=7.1 Hz, 2H), 2.17 (m, H), 1.03 (s, 3H), 1.01 (s, 3H) ppm. HR-MS(ESI): Calculated for C₂₅H₂₃N₆O₂ [M+H]⁺: 439.1882. found 439.1875.

The following compounds were prepared using a procedure similar to that in Example 1:

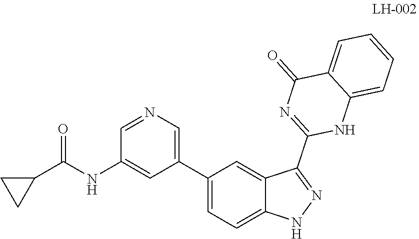

¹H NMR (300 MHz, DMSO-d₆) δ 14.25 (s, 1H), 12.27 (s, 1H), 10.69 (s, 1H), 8.91-8.82 (m, 2H), 8.66 (s, 1H), 8.42 (s, 1H), 8.14 (d, J=7.9 Hz, 1H), 7.76 (d, J=8.4 Hz, 3H), 7.62 (d, J=8.7 Hz, 1H), 7.39 (t, J=7.2 Hz, 1H), 1.90 (q, J=6.2 Hz, 1H), 0.91 (q, J=7.9, 5.5 Hz, 4H) ppm. HR-MS(ESI): Calculated for C₂₄H₁₉N₆O₂ [M+H]⁺: 423.1569. found 423.1562.

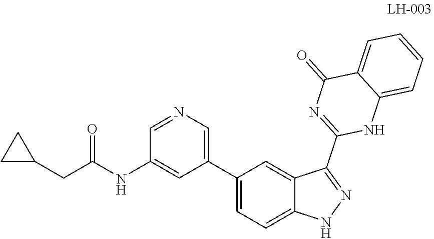

¹H NMR (300 MHz, DMSO-d₆) δ 14.28 (s, 1H), 12.29 (s, 1H), 9.80 (s, 1H), 9.61 (d, J=1.3 Hz, 1H), 8.83 (d, J=1.5 Hz, 1H), 8.70 (d, J=1.3 Hz, 1H), 8.13 (dd, J=7.4, 2.0 Hz, 1H), 8.03 (t, J=1.3 Hz, 1H), 7.71-7.55 (m, 3H), 7.45 (td, J=7.5, 2.0 Hz, 1H), 7.14 (td, J=7.5, 2.0 Hz, 1H), 2.10 (d, J=7.0 Hz, 2H), 1.15 (m, 1H), 0.62-0.44 (m, 2H), 0.32-0.27 (m, 2H) ppm. HR-MS(ESI): Calculated for C₂₅H₂₁N₆O₂ [M+H]⁺: 437.1726. found 437.1722.

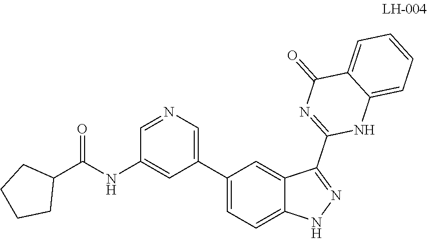

¹H NMR (300 MHz, DMSO-d₆) δ 14.16 (s, 1H), 10.56 (s, 1H), 8.93 (s, 2H), 8.67 (s, 1H), 8.20 (d, J=7.8 Hz, 1H), 7.95 (d, J=8.1 Hz, 1H), 7.88 (d, J=10.6 Hz, 3H), 7.56 (t, J=7.4 Hz, 1H), 5.78 (s, 2H), 2.91 (d, J=7.7 Hz, 1H), 1.99-1.57 (m, 8H) ppm. HR-MS(ESI): Calculated for C₂₆H₂₃N₆O₂ [M+H]⁺: 451.1882. found 451.1879.

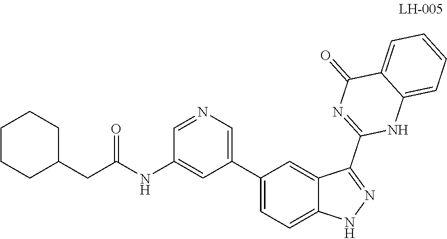

¹H NMR (300 MHz, DMSO-d₆) δ 14.43 (s, 1H), 11.66 (s, 1H), 10.31 (s, 1H), 8.90 (s, 1H), 8.81 (s, 1H), 8.70 (s, 1H), 8.53 (s, 1H), 8.21 (d, J=7.9 Hz, 1H), 7.88 (dd, J=7.7, 8.3 Hz, 4H), 7.55 (t, J=7.3 Hz, 1H), 2.32 (d, J=6.9 Hz, 2H), 1.86-1.66 (m, 6H), 1.25 (q, J=11.6, 10.8 Hz, 3H), 1.05 (q, J=11.3 Hz, 2H) ppm. HR-MS(ESI): Calculated for C₂₈H₂₇N₆O₂ [M+H]⁺: 479.2195. found 479.2191.

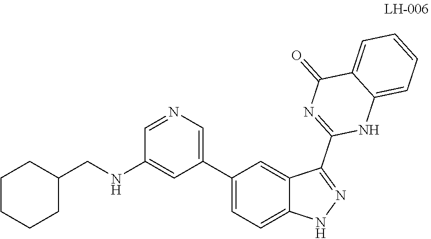

¹H NMR (300 MHz, DMSO-d₆) δ 14.08 (s, 1H), 12.21 (s, 1H), 8.83 (s, 1H), 8.22 (d, J=8.2 Hz, 2H), 8.04 (d, J=2.4 Hz, 1H), 7.89 (d, J=4.0 Hz, 2H), 7.82 (s, 2H), 7.57 (dt, J=8.2, 4.1 Hz, 1H), 7.38 (s, 1H), 3.05 (d, J=6.7 Hz, 2H), 1.88 (d, J=12.6 Hz, 2H), 1.77-1.60 (m, 5H), 1.26 (t, J=10.7 Hz, 3H), 1.02 (dt, J=12.0, 6.0 Hz, 2H) ppm. HR-MS(ESI): Calculated for C₂₇H₂₇N₆O [M+H]⁺: 451.2246. found 451.2241.

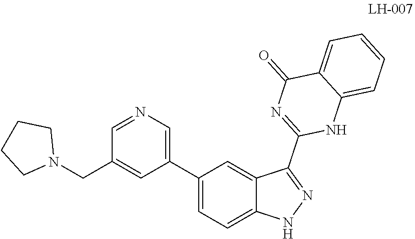

¹H NMR (300 MHz, DMSO-d₆) δ 14.11 (s, 1H), 12.22 (s, 1H), 8.90 (d, J=10.1 Hz, 2H), 8.58 (s, 1H), 8.21 (d, J=7.9 Hz, 1H), 8.12 (s, 1H), 7.86 (dd, J=14.2, 7.9 Hz, 4H), 7.57 (d, J=7.2 Hz, 1H), 3.86 (s, 2H), 2.64 (s, 4H), 1.80 (d, J=5.3 Hz, 4H) ppm. HR-MS(ESI): Calculated for C₂₅H₂₃N₆O [M+H]⁺: 423.1933. found 423.1928.

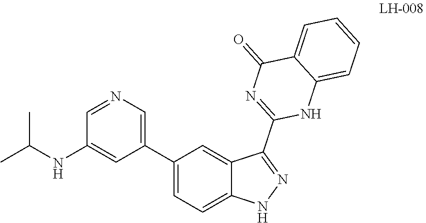

¹H NMR (300 MHz, DMSO-d₆) δ 14.15 (s, 1H), 12.63 (s, 1H), 8.81 (s, 1H), 8.21 (d, J=7.9 Hz, 1H), 8.15 (s, 1H), 8.01 (d, J=2.5 Hz, 1H), 7.87 (d, J=3.6 Hz, 2H), 7.79 (d, J=2.6 Hz, 2H), 7.55 (dq, J=8.0, 3.8, 3.2 Hz, 1H), 7.23 (s, 1H), 5.88 (d, J=7.9 Hz, 1H), 3.75 (d, J=6.8 Hz, 1H), 1.25 (s, 3H), 1.23 (s, 3H) ppm. HR-MS(ESI): Calculated for C₂₃H₂₁N₆O [M+H]⁺: 397.1777, found 397.1769.

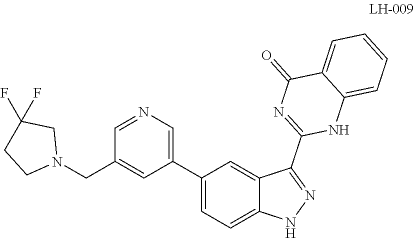

¹H NMR (300 MHz, DMSO-d₆) δ 14.14 (s, 1H), 12.21 (s, 1H), 8.96 (d, J=2.2 Hz, 1H), 8.89 (s, 1H), 8.60 (s, 1H), 8.24-8.13 (m, 2H), 7.88-7.69 (m, 4H), 7.59-7.52 (m, 1H), 3.92 (s, 2H), 3.11 (t, J=13.2 Hz, 2H), 2.94-2.88 (m, 2H), 2.43-2.36 (m, 2H) ppm. HR-MS(ESI): Calculated for C₂₅H₂₁F₂N₆O [M+H]⁺: 459.1745. found 459.1739.

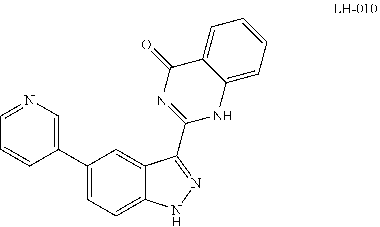

¹H NMR (300 MHz, DMSO-d₆) δ 14.27 (s, 1H), 12.28 (s, 1H), 9.00 (d, J=2.3 Hz, 1H), 8.85 (s, 1H), 8.65-8.58 (m, 1H), 8.23-8.12 (m, 2H), 7.83 (dd, J=9.9, 6.4 Hz, 3H), 7.73 (dd, J=8.7, 1.8 Hz, 1H), 7.56 (dd, J=8.0, 4.8 Hz, 1H), 7.49-7.42 (m, 1H) ppm. HR-MS(ESI): Calculated for C₂₀H₁₄N₅O [M+H]⁺: 340.1198. found 340.1192.

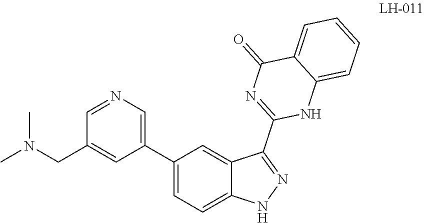

¹H NMR (300 MHz, DMSO-d₆) δ 14.28 (s, 1H), 12.14 (s, 1H), 8.89 (s, 2H), 8.52 (s, 1H), 8.17 (d, J=7.9 Hz, 1H), 8.05 (s, 1H), 7.84-7.69 (m, 4H), 7.45 (d, J=3.5 Hz, 1H), 3.58 (s, 2H), 2.25 (s, 6H) ppm. HR-MS(ESI): Calculated for C₂₃H₂₁N₆O [M+H]⁺: 397.1777. found 397.1771.

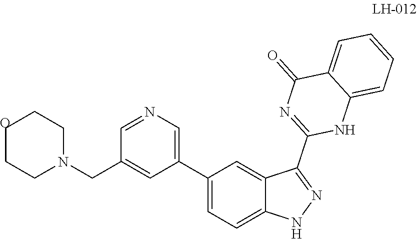

¹H NMR (300 MHz, DMSO-d₆) δ 14.09 (s, 1H), 12.25 (s, 1H), 8.91 (d, J=2.2 Hz, 1H), 8.87 (s, 1H), 8.57 (d, J=1.9 Hz, 1H), 8.21 (d, J=7.9 Hz, 1H), 8.12 (d, J=2.2 Hz, 1H), 7.93-7.81 (m, 6H), 7.57-7.51 (m, 1H), 3.72 (s, 2H), 3.64 (d, J=4.7 Hz, 6H) ppm. HR-MS(ESI): Calculated for C₂₅H₂₃N₆O₂ [M+H]⁺: 439.1882. found 439.1879.

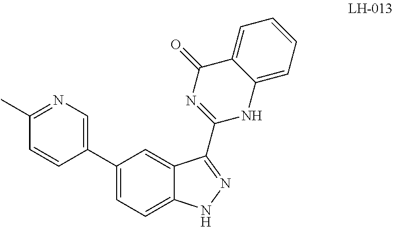

¹H NMR (300 MHz, DMSO-d₆) δ 14.11 (s, 1H), 12.22 (s, 1H), 8.61-8.47 (m, 3H), 8.19 (d, J=7.9 Hz, 1H), 7.91-7.78 (m, 3H), 7.55 (dt, J=11.2, 5.3 Hz, 2H), 7.44 (d, J=5.0 Hz, 1H), 2.36 (s, 3H) ppm. HR-MS(ESI): Calculated for C₂₁H₁₆N₅O [M+H]⁺: 354.1355. found 354.1348.

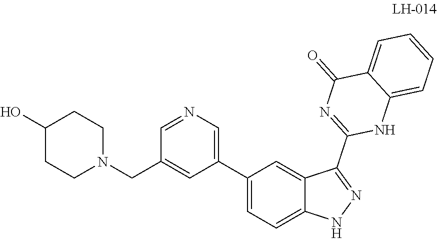

¹H NMR (300 MHz, DMSO-d₆) δ 14.11 (s, 1H), 12.29 (s, 1H), 8.92 (d, J=13.6 Hz, 2H), 8.59 (s, 1H), 8.22 (d, J=7.9 Hz, 2H), 7.91 (d, J=7.4 Hz, 4H), 7.57 (ddd, J=8.2, 6.0, 2.2 Hz, 1H), 3.77 (s, 1H), 3.65 (s, 2H), 1.81 (s, 2H), 1.53 (s, 3H), 1.25 (s, 3H) ppm. HR-MS(ESI): Calculated for C₂₆H₂₅N₆O₂ [M+H]⁺: 453.2039. found 453.2041.

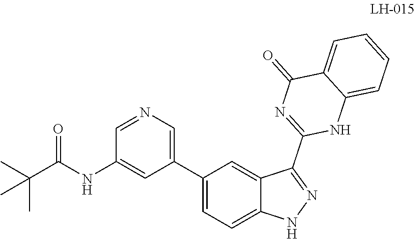

¹H NMR (300 MHz, DMSO-d₆) δ 14.14 (s, 1H), 12.20 (s, 1H), 9.68 (s, 1H), 8.98 (d, J=11.2 Hz, 2H), 8.77 (s, 1H), 8.60 (s, 1H), 8.29 (t, J=7.7 Hz, 1H), 8.04-7.89 (m, 4H), 7.63 (d, J=7.7 Hz, 1H), 1.44-1.36 (m, 9H) ppm. HR-MS(ESI): Calculated for C₂₅H₂₃N₆O₂ [M+H]⁺: 439.1882. found 439.1878.

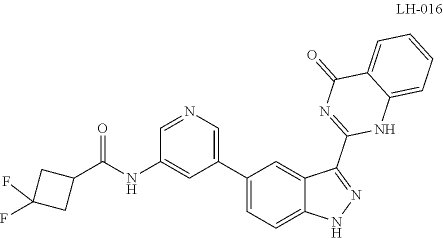 [SMILES: O=C(Nc1cncc(-c2ccc3[nH]nc(-c4nc(=O)c5ccccc5[nH]4)c3c2)c1)C1CC(F)(F)C1]

¹H NMR (300 MHz, DMSO-d₆) δ 14.21 (s, 1H), 12.29 (s, 1H), 10.85 (s, 1H), 8.89 (d, J=8.1 Hz, 2H), 8.73 (s, 1H), 8.56 (s, 1H), 8.21 (d, J=7.8 Hz, 1H), 7.91 (d, J=7.2 Hz, 4H), 7.63-7.50 (m, 1H), 2.93-2.85 (m, 3H), 1.23 (s, 2H) ppm. HR-MS(ESI): Calculated for C₂₅H₁₉F₂N₆O₂ [M+H]⁺: 473.1538. found 473.1532.

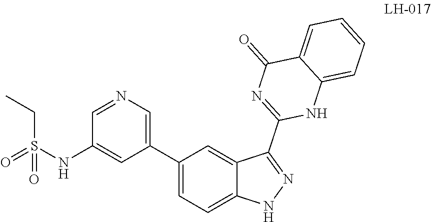 [SMILES: CCS(=O)(=O)Nc1cncc(-c2ccc3[nH]nc(-c4nc(=O)c5ccccc5[nH]4)c3c2)c1]

¹H NMR (300 MHz, DMSO-d₆) δ 14.14 (s, 1H), 12.31 (s, 1H), 10.34 (s, 1H), 8.92 (s, 1H), 8.79 (s, 1H), 8.52 (s, 1H), 8.22 (d, J=7.6 Hz, 1H), 8.05 (s, 1H), 7.90 (d, J=12.0 Hz, 4H), 7.58 (s, 1H), 3.31 (s, 2H), 1.25 (s, 3H) ppm. HR-MS(ESI): Calculated for C₂₂H₁₉N₆O₃S [M+H]⁺: 447.1239. found 447.1231.

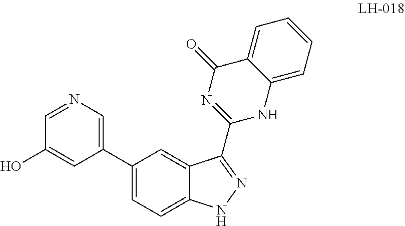 [SMILES: O=c1nc(-c2n[nH]c3ccc(-c4cncc(O)c4)cc23)[nH]c2ccccc12]

¹H NMR (300 MHz, DMSO-d₆) δ 14.73 (s, 1H), 12.25 (s, 1H), 8.84 (s, 1H), 8.39 (s, 1H), 8.14 (d, J=8.9 Hz, 2H), 7.74 (d, J=4.3 Hz, 3H), 7.63 (d, J=8.4 Hz, 1H), 7.52 (s, 1H), 7.39 (d, J=8.4 Hz, 1H) ppm. HR-MS(ESI): Calculated for C₂₀H₁₄N₅O₂ [M+H]⁺: 356.1147. found 356.1141.

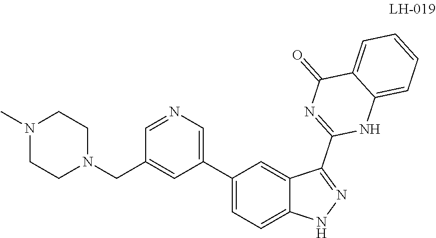 [SMILES: CN1CCN(Cc2cncc(-c3ccc4[nH]nc(-c5nc(=O)c6ccccc6[nH]5)c4c3)c2)CC1]

¹H NMR (300 MHz, DMSO-d₆) § 14.22 (s, 1H), 12.32 (s, 1H), 9.03-8.92 (m, 2H), 8.65 (d, J=1.9 Hz, 1H), 8.30 (d, J=7.9 Hz, 1H), 8.18 (t, J=2.1 Hz, 1H), 8.02-7.90 (m, 4H), 7.65-7.59 (m, 1H), 3.81 (s, 2H), 2.95 (s, 4H), 2.72 (s, 4H), 2.61 (s, 3H) ppm. HR-MS(ESI): Calculated for C₂₆H₂₆N₇O [M+H]⁺: 452.2199. found 452.2203.

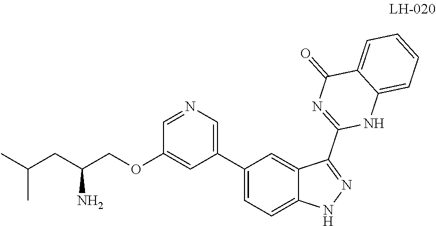 [SMILES: CC(C)C[C@H](N)COc1cncc(-c2ccc3[nH]nc(-c4nc(=O)c5ccccc5[nH]4)c3c2)c1]

¹H NMR (300 MHz, DMSO-d₆) δ 14.16 (s, 1H), 12.30 (s, 1H), 8.88 (s, 1H), 8.67 (d, J=1.7 Hz, 1H), 8.43 (d, J=2.6 Hz, 1H), 8.23-8.18 (m, 2H), 7.93-7.84 (m, 4H), 7.61-7.53 (m, 1H), 4.46 (dd, J=10.7, 3.2 Hz, 1H), 4.25 (dd, J=10.6, 6.7 Hz, 1H), 3.66 (s, 1H), 2.91 (s, 1H), 2.75 (s, 1H), 1.83-1.60 (m, 3H), 0.98 (d, J=1.9 Hz, 3H), 0.96 (d, J=2.0 Hz, 3H) ppm. HR-MS(ESI): Calculated for C₂₆H₂₇N₆O₂ [M+H]⁺: 455.2195. found 455.2191.

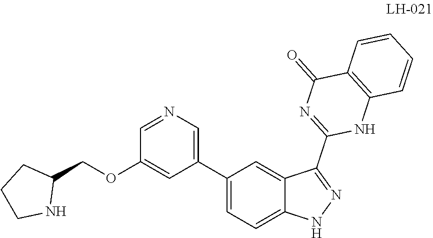

¹H NMR (300 MHz, DMSO-d₆) δ 14.36 (s, 1H), 12.18 (s, 1H), 8.57 (d, J=1.3 Hz, 1H), 8.22-8.08 (m, 2H), 7.72-7.52 (m, 4H), 7.45 (td, J=7.5, 2.0 Hz, 1H), 7.14 (td, J=7.5, 2.0 Hz, 1H), 4.33 (dd, J=12.4, 7.0 Hz, 1H), 4.16 (dd, J=12.4, 7.0 Hz, 1H), 3.25 (d, J=7.0 Hz, 1H), 2.97 (dt, J=9.4, 6.9 Hz, 1H), 2.79 (dt, J=9.3, 6.9 Hz, 1H), 1.77-1.65 (m, 3H), 1.41 (ddd, J=12.3, 7.6, 6.3 Hz, 1H), 1.19 (s, 1H) ppm. HR-MS(ESI): Calculated for C₂₅H₂₃N₆O₂ [M+H]⁺: 439.1882. found 439.1878.

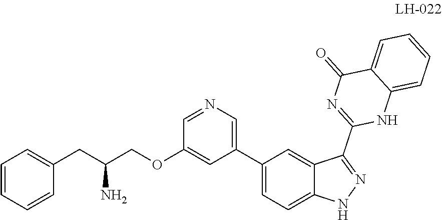 [SMILES: N[C@H](COc1cncc(-c2ccc3[nH]nc(-c4nc(=O)c5ccccc5[nH]4)c3c2)c1)Cc1ccccc1]

¹H NMR (300 MHz, DMSO-d₆) δ 14.05 (s, 1H), 12.53 (s, 1H), 8.78-8.71 (m, 1H), 8.59 (d, J=1.3 Hz, 1H), 8.18-8.05 (m, 2H), 7.71-7.51 (m, 4H), 7.44 (td, J=7.5, 2.0 Hz, 1H), 7.35-7.22 (m, 2H), 7.22-7.07 (m, 4H), 4.26 (dd, J=12.4, 7.0 Hz, 1H), 4.07 (dd, J=12.4, 6.9 Hz, 1H), 3.67-3.61 (m, 1H), 3.11-2.98 (m, 1H), 2.81-2.71 (m, 1H), 1.54 (s, 2H) ppm. HR-MS(ESI): Calculated for C₂₉H₂₅N₆O₂ [M+H]⁺: 4897.2039. found 489.2029.

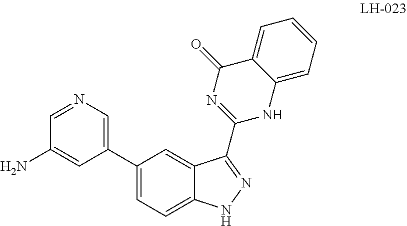 [SMILES: Nc1cncc(-c2ccc3[nH]nc(-c4nc(=O)c5ccccc5[nH]4)c3c2)c1]

¹H NMR (300 MHz, DMSO-d₆) δ 14.16 (s, 1H), 12.38 (s, 1H), 8.79 (d, J=1.1 Hz, 1H), 8.52 (d, J=1.3 Hz, 1H), 8.36 (d, J=1.3 Hz, 1H), 8.13 (dd, J=7.4, 2.0 Hz, 1H), 7.71-7.53 (m, 3H), 7.51-7.39 (m, 2H), 7.14 (td, J=7.5, 2.0 Hz, 1H), 3.50 (s, 2H) ppm. HR-MS(ESI): Calculated for C₂₀H₁₅N₆O [M+H]⁺: 355.1307. found 355.1301.

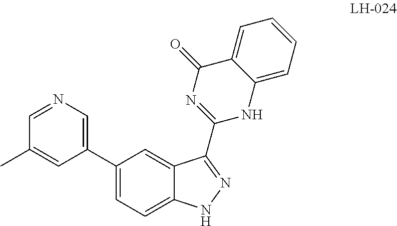 [SMILES: Cc1cncc(-c2ccc3[nH]nc(-c4nc(=O)c5ccccc5[nH]4)c3c2)c1]

¹H NMR (300 MHz, DMSO-d₆) § 14.42 (s, 1H), 12.27 (s, 1H), 8.83-8.73 (m, 2H), 8.48 (d, J=1.3 Hz, 1H), 8.13 (dd, J=7.4, 2.0 Hz, 1H), 7.89 (t, J=1.3 Hz, 1H), 7.66 (dd, J=7.5, 2.0 Hz, 1H), 7.63-7.50 (m, 2H), 7.45 (td, J=7.5, 2.0 Hz, 1H), 7.14 (td, J=7.5, 2.0 Hz, 1H), 2.48 (s, 3H) ppm. HR-MS(ESI): Calculated for C₂₁H₁₆N₅O [M+H]⁺: 354.1355. found 354.1350.

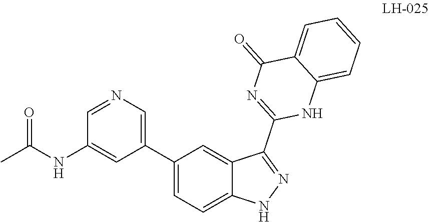 [SMILES: CC(=O)Nc1cncc(-c2ccc3[nH]nc(-c4nc(=O)c5ccccc5[nH]4)c3c2)c1]

¹H NMR (300 MHz, DMSO-d₆) δ 14.52 (s, 1H), 12.22 (s, 1H), 9.80 (s, 1H), 9.58 (d, J=1.3 Hz, 1H), 8.87-8.79 (m, 1H), 8.70 (d, J=1.3 Hz, 1H), 8.13 (dd, J=7.5, 2.0 Hz, 1H), 8.04 (t, J=1.3 Hz, 1H), 7.71-7.55 (m, 3H), 7.45 (td, J=7.5, 2.0 Hz, 1H), 7.14 (td, J=7.5, 2.0 Hz, 1H), 2.20 (s, 3H) ppm. HR-MS(ESI): Calculated for C₂₂H₁₇N₆O₂ [M+H]⁺: 397.1413. found 397.1410.

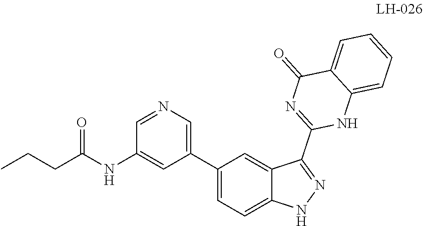 [SMILES: CCCC(=O)Nc1cncc(-c2ccc3[nH]nc(-c4nc(=O)c5ccccc5[nH]4)c3c2)c1]

¹H NMR (300 MHz, DMSO-d₆) δ 14.62 (s, 1H), 12.12 (s, 1H), 9.80 (s, 1H), 9.60 (d, J=1.3 Hz, 1H), 8.76 (d, J=1.6 Hz, 1H), 8.68 (d, J=1.3 Hz, 1H), 8.13 (dd, J=7.5, 2.1 Hz, 1H), 7.91 (t, J=1.3 Hz, 1H), 7.66 (dd, J=7.5, 2.0 Hz, 1H), 7.65-7.49 (m, 2H), 7.45 (td, J=7.5, 2.0 Hz, 1H), 7.14 (td, J=7.5, 2.0 Hz, 1H), 2.37 (t, J=8.1 Hz, 2H), 1.69-1.55 (m, 2H), 0.98 (t, J=8.0 Hz, 3H) ppm. HR-MS(ESI): Calculated for C₂₄H₂₁N₆O₂ [M+H]⁺: 425.1726. found 425.1720.

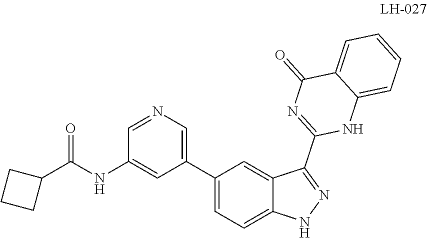 [SMILES: O=C(Nc1cncc(-c2ccc3[nH]nc(-c4nc(=O)c5ccccc5[nH]4)c3c2)c1)C1CCC1]

¹H NMR (300 MHz, DMSO-d₆) δ 14.55 (s, 1H), 12.14 (s, 1H), 9.80 (s, 1H), 9.62 (d, J=1.4 Hz, 1H), 8.76 (d, J=1.5 Hz, 1H), 8.68 (d, J=1.3 Hz, 1H), 8.13 (dd, J=7.5, 2.0 Hz, 1H), 7.91 (t, J=1.3 Hz, 1H), 7.71-7.55 (m, 2H), 7.54 (dd, J=7.5, 1.5 Hz, 1H), 7.45 (td, J=7.5, 2.0 Hz, 1H), 7.14 (td, J=7.5, 2.0 Hz, 1H), 3.22 (d, J=7.0 Hz, 1H), 2.55-2.37 (m, 2H), 2.17-1.82 (m, 4H) ppm. HR-MS(ESI): Calculated for C₂₅H₂₁N₆O₂ [M+H]⁺: 437.1726. found 437.1719.

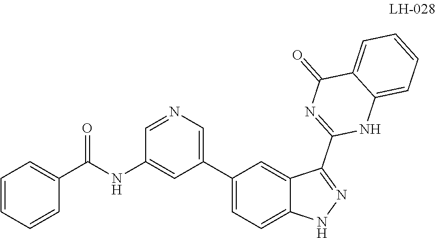 [SMILES: O=C(Nc1cncc(-c2ccc3[nH]nc(-c4nc(=O)c5ccccc5[nH]4)c3c2)c1)c1ccccc1]

¹H NMR (300 MHz, DMSO-d₆) § 14.25 (s, 1H), 12.25 (s, 1H), 9.50 (d, J=1.3 Hz, 1H), 8.86 (d, J=1.4 Hz, 1H), 8.71 (d, J=1.3 Hz, 1H), 8.14 (d, J=9.8 Hz, 2H), 8.06 (d, J=1.2 Hz, 1H), 8.02-7.90 (m, 2H), 7.71-7.40 (m, 6H), 7.31 (ddt, J=9.6, 7.2, 2.0 Hz, 1H), 7.15 (td, J=7.5, 2.1 Hz, 1H) ppm. HR-MS(ESI): Calculated for C₂₇H₁₉N₆O₂ [M+H]⁺: 459.1569. found 459.1560.

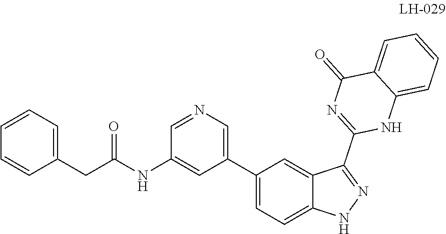 [SMILES: O=C(Cc1ccccc1)Nc1cncc(-c2ccc3[nH]nc(-c4nc(=O)c5ccccc5[nH]4)c3c2)c1]

¹H NMR (300 MHz, DMSO-d₆) δ 14.56 (s, 1H), 12.17 (s, 1H), 9.80 (s, 1H), 9.61 (d, J=1.2 Hz, 1H), 8.71 (dd, J=14.1, 1.4 Hz, 2H), 8.13 (dd, J=7.4, 2.0 Hz, 1H), 7.81 (t, J=1.3 Hz, 1H), 7.71-7.28 (m, 6H), 7.28-7.07 (m, 4H), 3.76 (s, 2H) ppm. HR-MS(ESI): Calculated for C₂₈H₂₁N₆O₂ [M+H]⁺: 473.1726. found 473.1719.

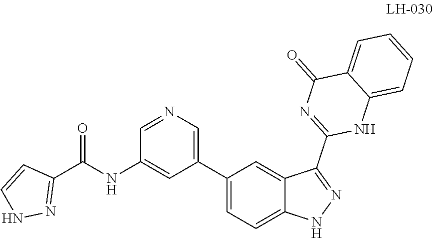 [SMILES: O=C(Nc1cncc(-c2ccc3[nH]nc(-c4nc(=O)c5ccccc5[nH]4)c3c2)c1)c1cc[nH]n1]

¹H NMR (300 MHz, DMSO-d₆) δ 14.16 (s, 1H), 12.12 (s, 1H), 12.35 (s, 1H), 9.53-9.42 (m, 2H), 8.83 (t, J=1.0 Hz, 1H), 8.70 (d, J=1.3 Hz, 1H), 8.19-8.02 (m, 2H), 7.71-7.55 (m, 3H), 7.45 (td, J=7.5, 2.0 Hz, 1H), 7.35 (d, J=7.5 Hz, 1H), 7.14 (td, J=7.5, 2.0 Hz, 1H), 6.86 (d, J=7.5 Hz, 1H) ppm. HR-MS(ESI): Calculated for C₂₄H₁₇N₈O₂ [M+H]⁺: 449.1474. found 449.1470.

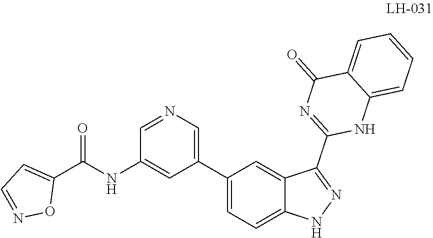 [SMILES: O=C(Nc1cncc(-c2ccc3[nH]nc(-c4nc(=O)c5ccccc5[nH]4)c3c2)c1)c1ccno1]

¹H NMR (300 MHz, DMSO-d₆) δ 14.11 (s, 1H), 12.27 (s, 1H), 9.48 (d, J=1.3 Hz, 2H), 8.90 (s, 1H), 8.80 (d, J=1.5 Hz, 2H), 8.71 (d, J=1.3 Hz, 2H), 8.13 (dd, J=7.4, 2.0 Hz, 2H), 7.96 (t, J=1.3 Hz, 2H), 7.67 (d, J=2.0 Hz, 1H), 7.45 (s, 1H) ppm. HR-MS(ESI): Calculated for C₂₄H₁₆N₇O₃ [M+H]⁺: 450.1315. found 450.1310.

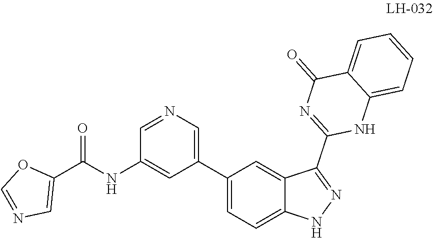 [SMILES: O=C(Nc1cncc(-c2ccc3[nH]nc(-c4nc(=O)c5ccccc5[nH]4)c3c2)c1)c1cnco1]

¹H NMR (300 MHz, DMSO-d₆) δ 14.11 (s, 1H), 12.27 (s, 1H), 9.49 (d, J=1.3 Hz, 1H), 8.90 (s, 1H), 8.79 (d, J=1.5 Hz, 1H), 8.70 (d, J=1.3 Hz, 1H), 8.13 (dd, J=7.5, 2.0 Hz, 1H), 8.06 (s, 1H), 7.95 (t, J=1.3 Hz, 1H), 7.71-7.50 (m, 3H), 7.45 (td, J=7.5, 2.0 Hz, 1H), 7.39 (s, 1H), 7.14 (td, J=7.5, 2.0 Hz, 1H) ppm. HR-MS(ESI): Calculated for C₂₄H₁₆N₇O₃ [M+H]⁺: 450.1315. found 450.1309.

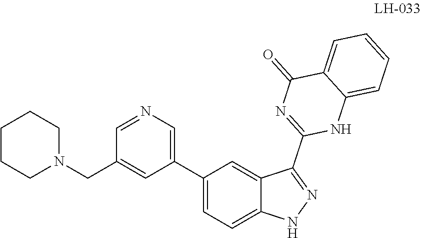 [SMILES: O=c1nc(-c2n[nH]c3ccc(-c4cncc(CN5CCCCC5)c4)cc23)[nH]c2ccccc12]

¹H NMR (300 MHz, DMSO-d₆) δ 14.14 (s, 1H), 12.25 (s, 1H), 8.88-8.74 (m, 2H), 8.71 (d, J=1.3 Hz, 1H), 8.23-8.13 (m, 2H), 7.71-7.51 (m, 3H), 7.45 (td, J=7.5, 2.0 Hz, 1H), 7.14 (td, J=7.5, 2.0 Hz, 1H), 3.69 (s, 2H), 2.43 (t, J=5.3 Hz, 4H), 1.62-1.36 (m, 6H) ppm. HR-MS(ESI): Calculated for C₂₆H₂₅N₆O [M+H]⁺: 437.2090. found 437.2088.

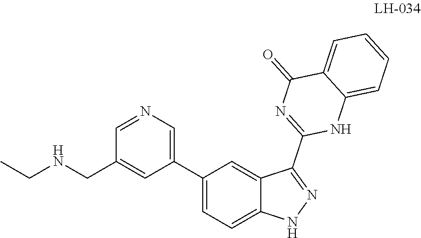 [SMILES: CCNCc1cncc(-c2ccc3[nH]nc(-c4nc(=O)c5ccccc5[nH]4)c3c2)c1]

¹H NMR (300 MHz, DMSO-d₆) δ 14.63 (s, 1H), 12.32 (s, 1H), 8.86-8.70 (m, 3H), 8.18-8.04 (m, 2H), 7.71-7.51 (m, 3H), 7.45 (td, J=7.5, 2.0 Hz, 1H), 7.14 (td, J=7.5, 2.0 Hz, 1H), 3.65 (s, 2H), 2.59 (q, J=8.0 Hz, 2H), 1.37 (s, 1H), 1.11 (t, J=8.0 Hz, 3H) ppm. HR-MS(ESI): Calculated for C₂₃H₂₁N₆O [M+H]⁺: 397.1777. found 397.1770.

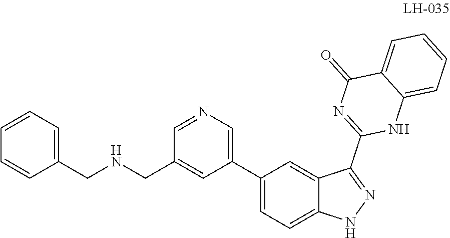 [SMILES: O=c1nc(-c2n[nH]c3ccc(-c4cncc(CNCc5ccccc5)c4)cc23)[nH]c2ccccc12]

¹H NMR (300 MHz, DMSO-d₆) & 14.51 (s, 1H), 12.31 (s, 1H), 8.84-8.74 (m, 2H), 8.65 (d, J=1.3 Hz, 1H), 8.20-8.09 (m, 2H), 7.71-7.56 (m, 3H), 7.51-7.20 (m, 6H), 7.14 (td, J=7.5, 2.0 Hz, 1H), 3.65 (s, 4H), 1.69 (s, 1H) ppm. HR-MS(ESI): Calculated for C₂₈H₂₃N₆O [M+H]⁺: 459.1933. found 459.1926.

### Example 2: Synthesis of LH-036

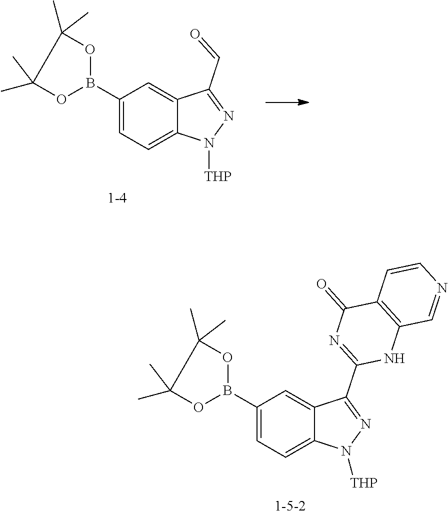

Intermediate 1-4 (0.1 mmol) was added to a single-necked flask and dissolved with 5 mL of dimethylacetamide. 3-Aminopyridine-4-carboxamide (0.15 mmol), p-toluenesulfonic acid hydrate (0.15 mmol), and sodium bisulfite (0.15 mmol) were then added, and the mixture was reacted at 120° C. for 6-8 h. After the TLC reaction was completed, the reaction mixture was extracted with ethyl acetate and saturated brine three times. The organic phases were combined, dried, concentrated, and purified by column chromatography to give intermediate 1-5-2. ¹H NMR (300 MHz, DMSO-d₆) δ 12.31 (s, 1H), 9.47 (s, 1H), 8.41-8.27 (m, 2H), 8.13 (d, J=1.6 Hz, 1H), 7.56 (d, J=7.5 Hz, 1H), 7.37 (dd, J=7.5, 1.6 Hz, 1H), 6.31 (t, J=6.9 Hz, 1H), 3.90 (dt, J=11.4, 7.0 Hz, 1H), 3.77 (dt, J=11.4, 7.0 Hz, 1H), 2.52-2.32 (m, 1H), 2.12-1.91 (m, 2H), 1.86-1.48 (m, 3H), 1.42 (d, J=14.9 Hz, 12H) ppm. HR-MS(ESI): Calculated for C₂₅H₂₉BN₅O₄ [M+H]⁺: 474.2313. found 474.2310. The yield was 75%.

The other steps were the same as those in Example 1 to give compound LH-036.

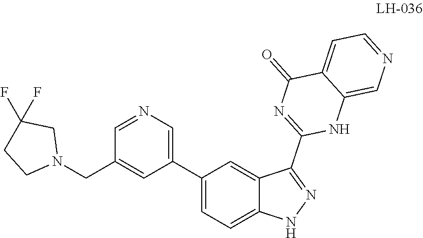 [SMILES: O=c1nc(-c2n[nH]c3ccc(-c4cncc(CN5CCC(F)(F)C5)c4)cc23)[nH]c2cnccc12]

¹H NMR (300 MHz, DMSO-d₆) δ 14.19 (s, 1H), 12.66 (s, 1H), 9.29 (s, 1H), 8.97 (s, 1H), 8.90 (s, 1H), 8.69 (d, J=5.1 Hz, 1H), 8.61 (s, 1H), 8.18 (s, 1H), 8.02 (d, J=5.1 Hz, 1H), 7.88 (q, J=8.7 Hz, 2H), 3.95 (s, 2H), 3.17-3.08 (m, 2H), 2.93 (t, J=6.9 Hz, 2H), 2.38 (dq, J=15.3, 7.9, 7.4 Hz, 2H) ppm. HR-MS(ESI): Calculated for C₂₄H₂₀F₂N₇O [M+H]⁺: 460.1697. found 460.1690.

The following compounds were prepared using a procedure similar to that in Example 2:

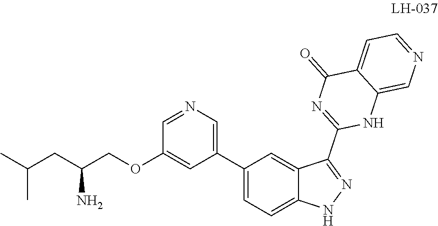 [SMILES: CC(C)C[C@H](N)COc1cncc(-c2ccc3[nH]nc(-c4nc(=O)c5ccncc5[nH]4)c3c2)c1]

¹H NMR (300 MHz, DMSO-d₆) δ 14.55 (s, 1H), 12.33 (s, 1H), 9.47 (s, 1H), 8.76 (t, J=1.0 Hz, 1H), 8.65 (d, J=1.3 Hz, 1H), 8.40 (d, J=5.0 Hz, 1H), 8.31 (d, J=5.0 Hz, 1H), 8.22 (d, J=1.4 Hz, 1H), 7.61 (dd, J=16.8, 1.2 Hz, 3H), 4.33 (dd, J=12.4, 7.0 Hz, 1H), 4.08 (dd, J=12.4, 7.0 Hz, 1H), 3.23-3.13 (m, 1H), 1.73-1.58 (m, 2H), 1.58-1.40 (m, 1H), 1.34 (s, 2H), 0.99-0.82 (m, 6H) ppm. HR-MS(ESI): Calculated for C₂₅H₂₆N₇O₂ [M+H]⁺: 456.2148. found 456.2140.

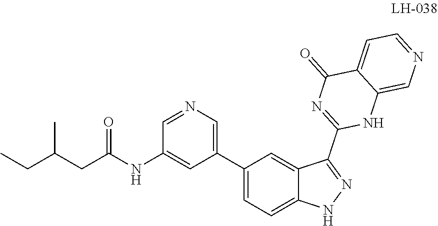 [SMILES: CCC(C)CC(=O)Nc1cncc(-c2ccc3[nH]nc(-c4nc(=O)c5ccncc5[nH]4)c3c2)c1]

¹H NMR (300 MHz, DMSO-d₆) δ 14.34 (s, 1H), 12.21 (s, 1H), 9.80 (s, 1H), 9.64 (d, J=1.4 Hz, 1H), 9.47 (s, 1H), 8.82-8.70 (m, 2H), 8.40 (d, J=5.0 Hz, 1H), 8.31 (d, J=5.0 Hz, 1H), 8.00 (s, 1H), 7.61 (d, J=1.0 Hz, 2H), 2.22-2.05 (m, 3H), 1.08-0.93 (m, 6H) ppm. HR-MS(ESI): Calculated for C₂₄H₂₂N₇O₂ [M+H]⁺: 440.1835. found 440.1835.

### Example 3: Synthesis of LH-039

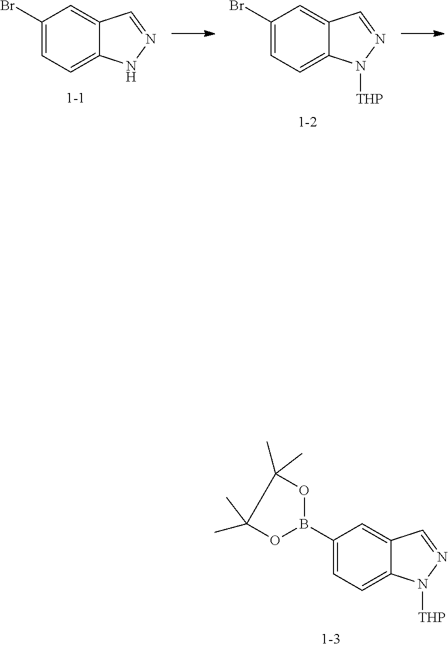

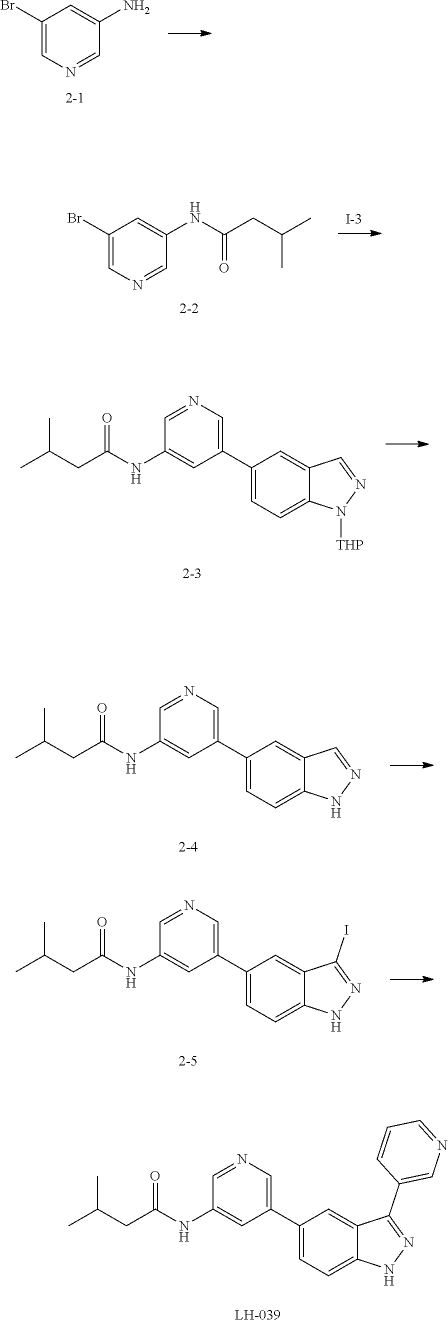

### Synthesis of Intermediate 1-2:

Intermediate 1-1 (0.1 mmol) was added to a three-necked flask. Tetrahydrofuran (50 mL) and p-toluenesulfonic acid (0.02 mmol) were added, and 3,4-dihydropyran (0.2 mmol) was added dropwise at room temperature. The mixture was reacted at 65° C. for 8 h. After the TLC reaction was completed, the reaction mixture was concentrated by rotary evaporation under reduced pressure to remove the solvent, and purified by silica gel column chromatography to give white solid I-2. ¹H NMR (300 MHz, DMSO-d₆) δ 7.97 (d, J=1.5 Hz, 1H), 7.89 (s, 1H), 7.40-7.27 (m, 2H), 6.27-6.16 (m, 1H), 3.90 (dt, J=11.4, 7.0 Hz, 1H), 3.77 (dt, J=11.5, 7.0 Hz, 1H), 2.38-2.16 (m, 1H), 2.04-2.01 (m, 2H), 1.84-1.65 (m, 1H), 1.58-1.32 (m, 2H) ppm. HR-MS(ESI): Calculated for C₁₂H₁₄BrN₂O [M+H]⁺: 281.0290. found 281.0292. The yield was 85%.

### Synthesis of Intermediate 1-3:

Intermediate 1-2 (0.1 mmol) was added to a single-necked flask, and bis(pinacolato)diboron (0.12 mmol), potassium acetate (0.3 mmol), Pd(dppf) C₁₂ (0.005 mmol), and dioxane (26 mL) were added. The mixture was warmed at reflux for 6 h. After the TLC reaction was completed, the reaction mixture was cooled, filtered under vacuum, concentrated by rotary evaporation, and purified by silica gel column chromatography to give white solid 1-3. ¹H NMR (300 MHz, DMSO-d₆) δ 7.97 (d, J=1.5 Hz, 1H), 7.82-7.74 (m, 1H), 7.39 (d, J=7.5 Hz, 1H), 7.25 (dd, J=7.5, 1.5 Hz, 1H), 6.21 (t, J=6.9 Hz, 1H), 3.90 (dt, J=11.4, 7.0 Hz, 1H), 3.77 (dt, J=11.4, 7.0 Hz, 1H), 2.40-2.21 (m, 1H), 2.16-1.91 (m, 2H), 1.85-1.48 (m, 3H), 1.40 (d, J=15.1 Hz, 12H) ppm. HR-MS(ESI): Calculated for C₁₈H₂₆BN₂O₃ [M+H]⁺: 329.2036. found 329.2033. The yield was 89%.

### Synthesis of Intermediate 2-2:

Starting material 2-1 (0.1 mmol) was dissolved in tetrahydrofuran. Triethylamine (0.15 mmol) and isovaleric anhydride (0.15 mmol) were added, and the mixture was warmed at reflux for 6-8 h. After the reaction was completed as detected by TLC, the reaction mixture was concentrated under reduced pressure to remove the solvent, the reaction solution was poured into an ice saturated sodium hydroxide solution to remove excess anhydride, and the mixture was extracted with ethyl acetate and saturated brine three times. The organic phases were combined, dried, and concentrated to give yellow powdery solid 2-2. ¹H NMR (300 MHz, DMSO-d₆) δ 9.80 (s. 1H), 9.58 (d, J=1.3 Hz, 1H), 8.49 (d, J=1.3 Hz, 1H), 7.87 (t, J=1.3 Hz, 1H), 2.24 (m, 1H), 2.10 (d, J=6.9 Hz, 2H), 0.95 (d, J=6.7 Hz, 6H) ppm. HR-MS(ESI): Calculated for C₁₀H₁₄BrN₂O [M+H]⁺: 257.0290. found 257.0286. The yield was 85%.

### Synthesis of Intermediate 2-3:

Intermediate 2-2 (0.1 mmol), intermediate 1-3 (0.1 mmol), sodium carbonate (0.3 mmol), Pd(dppf)Cl₂ (0.005 mmol), dioxane (9 mL), and water (1.5 mL) were added to a single-necked flask, and the system was purged of air with nitrogen 4 times. The mixture was warmed at reflux for 8 h. After the mixture was cooled, the mixture was filtered under vacuum, concentrated to dryness by rotary evaporation, and purified by silica gel column chromatography to give intermediate 2-3. ¹H NMR (300 MHz, DMSO-d₆) δ 9.80 (s, 1H), 9.63 (d, J=1.3 Hz, 1H), 8.69 (d, J=1.3 Hz, 1H), 8.19 (t, J=1.6 Hz, 1H), 8.03 (d, J=1.5 Hz, 1H), 7.90 (t, J=1.3 Hz, 1H), 7.68 (d, J=7.5 Hz, 1H), 7.51 (dd, J=7.5, 1.5 Hz, 1H), 6.25 (t, J=6.9 Hz, 1H), 3.90 (dt, J=11.5, 7.0 Hz, 1H), 3.77 (dt, J=11.5, 7.0 Hz, 1H), 2.64-2.57 (qd, J=12.4, 7.0 Hz, 2H), 2.39-2.13 (m, 2H), 2.13-1.91 (m, 2H), 1.85-1.48 (m, 3H), 0.95 (dd, J=15.0, 6.8 Hz, 6H) ppm. HR-MS(ESI): Calculated for C₂₂H₂₇N₄O₂ [M+H]⁺: 379.2134. found 379.2132. The yield was 78%.

### Synthesis of Intermediate 2-4:

Intermediate 2-3 (0.1 mmol) was dissolved in 10 mL of tetrahydrofuran. p-Toluenesulfonic acid hydrate (1 mmol) was added at room temperature, and the mixture was warmed at reflux for 16-24 h. After the reaction was completed as detected by TLC, the reaction mixture was concentrated under reduced pressure and extracted with dichloromethane and saturated sodium bicarbonate. The organic phases were combined, dried, and concentrated to give intermediate 2-4. ¹H NMR (300 MHz, DMSO-d₆) δ 12.75 (s, 1H), 9.80 (s, 1H), 9.63 (d, J=1.3 Hz, 1H), 8.50 (d, J=1.3 Hz, 1H), 8.06 (d, J=1.6 Hz, 1H), 7.96 (t, J=1.5 Hz, 1H), 7.61 (t, J=1.3 Hz, 1H), 7.43-7.24 (m, 2H), 2.24-2.05 (m, 3H), 0.95 (d, J=6.3 Hz, 6H) ppm. HR-MS(ESI): Calculated for C₁₇H₁₉N₄O [M+H]⁺: 295.1559. found 295.1554. The yield was 74%.

### Synthesis of Intermediate 2-5:

Intermediate 2-3 (0.1 mmol) was dissolved in 5 mL of dimethylformamide. Potassium hydroxide (0.15 mmol) and iodine (0.15 mmol) were added, and the mixture was stirred at room temperature for 16-24 h. After the reaction was completed as detected by TLC, the reaction mixture was extracted with ethyl acetate and saturated brine three times. The organic phases were combined, dried, and concentrated to give the intermediate 2-5. ¹H NMR (300 MHz, DMSO-d₆) δ 12.35 (s, 1H), 9.64 (s, 1H), 9.25 (d, J=1.3 Hz, 1H), 8.57 (d, J=1.3 Hz, 1H), 7.23 (t, J=1.5 Hz, 1H), 7.4 (t, J=1.3 Hz, 1H), 7.40-7.22 (m, 2H), 2.24-2.05 (m, 3H), 0.82 (d, J=6.3 Hz, 6H) ppm. HR-MS(ESI): Calculated for C₁₇H₁₈IN₄O [M+H]⁺: 421.0525. found 421.0525. The yield was 65%.

### Synthesis of LH-039:

Intermediate 2-5 (0.1 mmol), 3-pyridineboronic acid (0.1 mmol), sodium carbonate (0.3 mmol), Pd(dppf)Cl₂ (0.005 mmol), dioxane (9 mL), and water (1.5 mL) were added to a single-necked flask, and the system was purged of air with nitrogen 4 times. The mixture was warmed at reflux for 8 h. After the mixture was cooled, the mixture was filtered under vacuum, concentrated to dryness by rotary evaporation, and purified by silica gel column chromatography to give LH-039. ¹H NMR (300 MHz, DMSO-d₆) δ 13.62 (s, 1H), 10.23 (s, 1H), 9.30 (d, J=2.2 Hz, 1H), 8.82 (d, J=2.3 Hz, 1H), 8.74 (d, J=2.1 Hz, 1H), 8.67 (dd, J=4.8, 1.6 Hz, 1H), 8.49 (dt, J=7.9, 2.0 Hz. 1H), 8.40-8.32 (m, 2H), 7.83-7.73 (m, 2H), 7.60 (dd, J=8.0, 4.8 Hz, 1H), 2.29 (d, J=7.1 Hz. 2H), 2.14 (dq, J=13.5, 6.6 Hz, 1H), 1.00 (s, 3H), 0.98 (s, 3H) ppm. HR-MS(ESI): Calculated for C₂₂H₂₂N₅O [M+H]⁺: 372.1824. found 372.1818.

The following compounds were prepared using a procedure similar to that in Example 3:

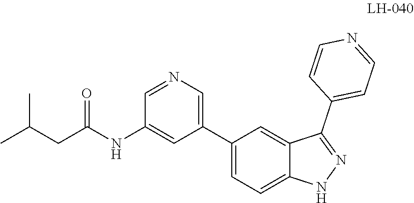 [SMILES: CC(C)CC(=O)Nc1cncc(-c2ccc3[nH]nc(-c4ccncc4)c3c2)c1]

¹H NMR (300 MHz, DMSO-d₆) δ 13.74 (s, 1H), 10.22 (s, 1H), 8.79 (t, J=3.3 Hz, 1H), 8.70 (dd, J=4.9, 1.8 Hz, 3H), 8.40-8.30 (m, 2H), 8.11-8.01 (m, 2H), 7.81-7.66 (m, 2H), 2.25 (d, J=7.1 Hz, 2H), 2.11 (dt, J=13.7, 6.8 Hz, 1H), 0.96 (s, 3H), 0.93 (d, J=4.7 Hz, 3H) ppm. HR-MS(ESI): Calculated for C₂₂H₂₂NO [M+H]⁺: 372.1824. found 372.1819.

### Example 4: Synthesis of LH-041

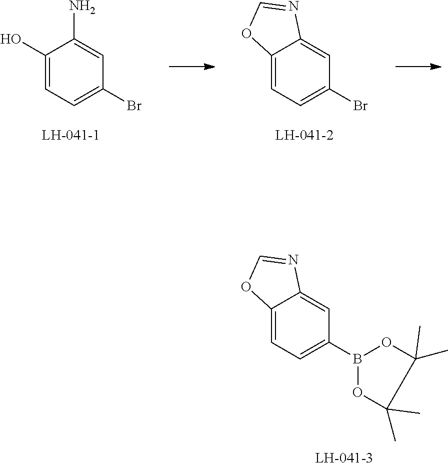 [SMILES: Nc1cc(Br)ccc1O ; Brc1ccc2ocnc2c1 ; CC1(C)OB(c2ccc3ocnc3c2)OC1(C)C]

Starting material LH-041-1 (2-amino-4-bromophenol, 0.1 mmol) was dissolved in triethyl orthoformate. The mixture was refluxed overnight, concentrated under reduced pressure, and purified by column chromatography to give intermediate LH-041-2. ¹H NMR (300 MHz, DMSO-d₆) δ 8.25 (s, 1H), 7.58 (d, J=1.5 Hz, 1H), 7.49 (dd, J=7.5, 1.6 Hz, 1H), 7.25 (d, J=7.5 Hz, 1H) ppm. HR-MS(ESI): Calculated for C₇H₅BrNO [M+H]⁺: 197.9555. found 197.9523. The yield was 57%.

Intermediate LH-041-2 (0.1 mmol) was added to a single-necked flask, and bis(pinacolato)diboron (0.12 mmol), potassium acetate (0.3 mmol), Pd(dppf)Cl₂ (0.005 mmol), and dioxane (25 mL) were added. The mixture was warmed at reflux for 6 h. After the TLC reaction was completed, the reaction mixture was cooled, filtered under vacuum, concentrated by rotary evaporation, and purified by silica gel column chromatography to give intermediate LH-041-3. ¹H NMR (300 MHz, DMSO-d₆) δ 8.25 (s, 1H), 7.75 (dd, J=7.5, 1.5 Hz, 1H), 7.58 (d, J=7.4 Hz, 1H), 7.48 (d, J=1.5 Hz, 1H), 1.40 (s. 12H) ppm. HR-MS(ESI): Calculated for C₁₃H₁₇BNO₃ [M+H]⁺: 246.1301. found 246.1281. The yield was 88%.

The other steps were the same as those in Example 3 to give compound LH-041.

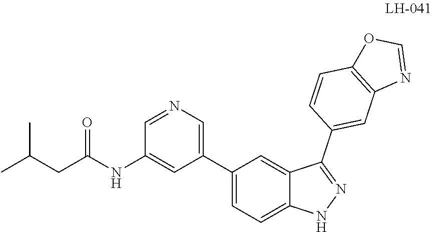 [SMILES: CC(C)CC(=O)Nc1cncc(-c2ccc3[nH]nc(-c4ccc5ocnc5c4)c3c2)c1]

¹H NMR (300 MHz, DMSO-d₆) δ 13.44 (s, 1H), 10.19 (s, 1H), 8.83 (s, 1H), 8.78 (d, J=2.3 Hz, 1H), 8.70 (d, J=2.1 Hz, 1H), 8.41 (d, J=1.6 Hz, 1H), 8.34 (t, J=2.2 Hz, 1H), 8.30 (d, J=1.4 Hz, 1H), 8.14 (dd, J=8.5, 1.7 Hz, 1H), 7.92 (dd, J=8.5, 0.6 Hz, 1H), 7.78-7.67 (m, 1H), 6.54 (s, 1H), 2.25 (d, J=7.1 Hz, 2H), 2.18-2.04 (m, 1H), 0.96 (s, 3H), 0.94 (s, 3H) ppm. HR-MS(ESI): Calculated for C₂₄H₂₂N₅O₂ [M+H]⁺: 412.1773. found 412.1769.

### Example 5: Synthesis of LH-042

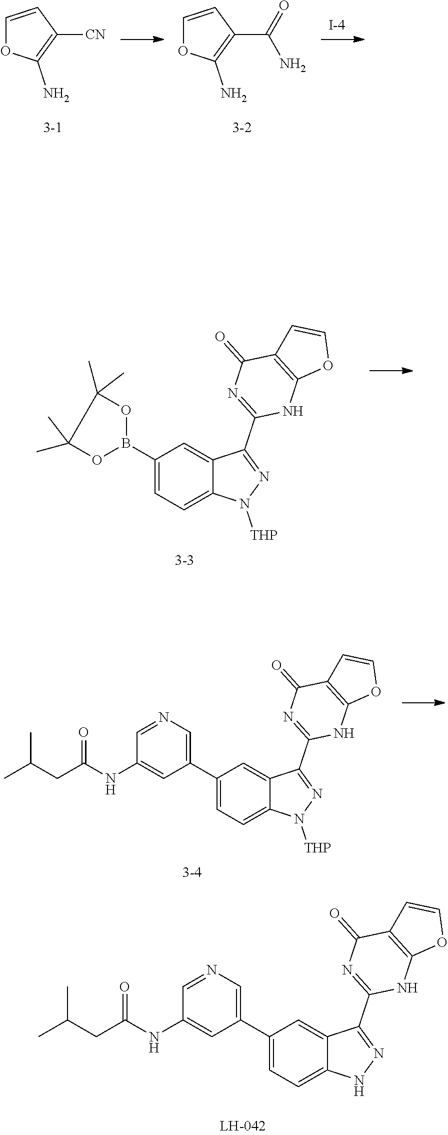

### Synthesis of Intermediate 3-2:

3-1 (0.1 mmol) was added into a single-necked flask, excess concentrated sulfuric acid was then added, and the mixture was stirred at room temperature for 2 h. After the reaction was completed, the reaction system was cooled to 0° C., and the mixture was alkalified with a 10% sodium hydroxide solution and extracted with ethyl acetate. The combined extracts were dried over magnesium sulfate, and the solvent was removed under vacuum. Recrystallization in ethanol was then performed to give 3-2.

### Synthesis of Intermediate 3-3:

Intermediate 3-2 (0.1 mmol) was added to a single-necked flask and dissolved with ethanol. Intermediate 1-4 in Example 1 (0.1 mmol) and iodine (0.1 mmol) were then added, and the mixture was refluxed at 80° C. for 8 h. After the TLC reaction was completed, 5% sodium thiosulfate was added to quench the reaction, and the mixture was extracted with dichloro and saturated brine. The organic phases were combined, dried, concentrated, and purified by column chromatography to give intermediate 3-3.

### Synthesis of Intermediate 3-4:

Intermediate 2-2 in Example 1 (0.1 mmol), intermediate 3-3 (0.1 mmol), sodium carbonate (0.3 mmol), Pd(dppf)Cl₂ (0.005 mmol), dioxane (9 mL), and water (1.5 mL) were added to a single-necked flask, and the system was purged of air with nitrogen 4 times. The mixture was warmed at reflux for 8 h. After the mixture was cooled, the mixture was filtered under vacuum, concentrated to dryness by rotary evaporation, and purified by silica gel column chromatography to give intermediate 3-4.

### Synthesis of LH-042:

Intermediate 3-4 (0.1 mmol) was added into a 50 mL single-necked flask and dissolved with 10 mL of dichloromethane. Excess trifluoroacetic acid (1 mmoL) was added, and the mixture was stirred at room temperature overnight. After the reaction was completed as monitored by TLC, the reaction mixture was concentrated under vacuum to remove the solvent and excess trifluoroacetic acid, mixed with silica gel, and purified by column chromatography to give LH-042. ¹H NMR (300 MHz, DMSO-d₆) δ 14.26 (s, 1H), 12.20 (s, 1H), 10.12 (s, 1H), 9.80 (s, 1H), 9.59 (d, J=1.3 Hz, 1H), 8.75 (d, J=1.5 Hz, 1H), 8.68 (d, J=1.3 Hz, 1H), 8.36 (s, 1H), 7.91 (t, J=1.2 Hz, 1H), 7.64-7.49 (m, 2H), 6.88 (q, J=7.5 Hz, 2H), 2.53-2.32 (m, 1H), 2.10 (d, J=7.0 Hz, 2H), 0.96 (d, J=6.7 Hz, 6H) ppm. HR-MS(ESI): Calculated for C₂₃H₂₁N₆O₃ [M+H]⁺: 429.1675. found 429.1670.

The following compounds were prepared using a procedure similar to that in Example 5:

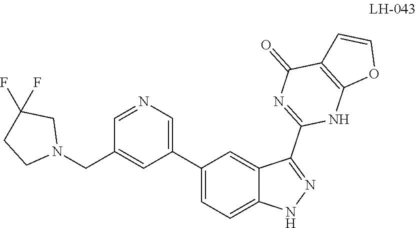 [SMILES: O=c1nc(-c2n[nH]c3ccc(-c4cncc(CN5CCC(F)(F)C5)c4)cc23)[nH]c2occc12]

¹H NMR (300 MHz, DMSO-d₆) δ 12.64 (s, 1H), 10.34 (s, 1H), 8.83 (d, J=1.3 Hz, 1H), 8.74 (dd, J=6.1, 1.5 Hz, 2H), 8.08 (t, J=1.3 Hz, 1H), 7.63-7.50 (m, 2H), 7.31 (d, J=7.5 Hz, 1H), 6.98 (d, J=7.5 Hz, 1H), 3.69 (s, 2H), 2.70-2.55 (m, 4H), 1.91-1.87 (m, 2H) ppm. HR-MS(ESI): Calculated for C₂₃H₁₉F₂N₆O₂ [M+H]⁺: 449.1538. found 449.1528.

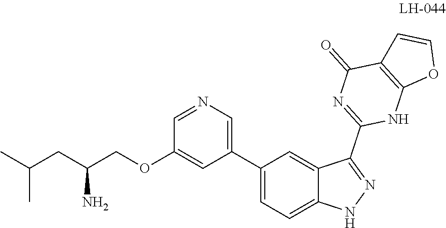 [SMILES: CC(C)C[C@H](N)COc1cncc(-c2ccc3[nH]nc(-c4nc(=O)c5ccoc5[nH]4)c3c2)c1]

¹H NMR (300 MHz, DMSO-d₆) § 12.12 (s, 1H), 10.15 (s, 1H), 8.79-8.71 (m, 1H), 8.60 (d, J=1.3 Hz, 1H), 8.18 (d, J=1.4 Hz, 1H), 7.60 (dt, J=16.7, 1.4 Hz, 3H), 7.31 (d, J=7.5 Hz, 1H), 6.98 (d, J=7.6 Hz, 1H), 4.32 (dd, J=12.4, 7.0 Hz, 1H), 4.09 (dd, J=12.4, 7.0 Hz, 1H), 3.22-3.05 (m, 1H), 1.73-1.41 (m, 3H), 1.54 (s, 2H), 0.99-0.81 (m, 6H) ppm. HR-MS(ESI): Calculated for C₂₄H₂₅N₆O₃ [M+H]⁺: 445.1988. found 445.1976.

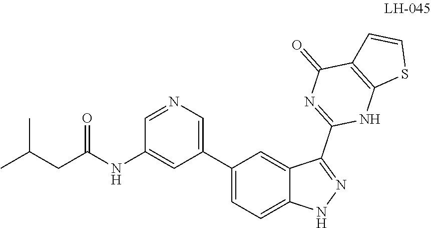 [SMILES: CC(C)CC(=O)Nc1cncc(-c2ccc3[nH]nc(-c4nc(=O)c5ccsc5[nH]4)c3c2)c1]

¹H NMR (300 MHz, DMSO-d₆) δ 13.24 (s, 1H), 10.31 (s, 1H), 9.80 (s, 1H), 9.60 (d, J=1.3 Hz, 1H), 8.76 (d, J=1.5 Hz, 1H), 8.68 (d, J=1.3 Hz, 1H), 7.91 (t, J=1.2 Hz, 1H), 7.65-7.49 (m, 2H), 7.36 (d, J=7.6 Hz, 1H), 6.85 (d, J=7.6 Hz, 1H), 2.32 (dt, J=13.6, 6.8 Hz, 1H), 2.10 (d, J=7.0 Hz, 2H), 0.96 (d, J=6.8 Hz, 6H) ppm. HR-MS(ESI): Calculated for C₂₃H₂₁N₆O₂S [M+H]⁺: 445.1447. found 445.1438.

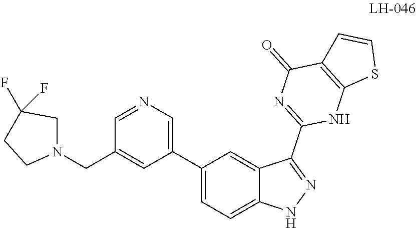 [SMILES: O=c1nc(-c2n[nH]c3ccc(-c4cncc(CN5CCC(F)(F)C5)c4)cc23)[nH]c2sccc12]

¹H NMR (300 MHz, DMSO-d₆) δ 13.26 (s, 1H), 10.18 (s, 1H), 8.84 (d, J=1.4 Hz, 1H), 8.81-8.68 (m, 2H), 8.10 (t, J=1.3 Hz, 1H), 7.64-7.50 (m, 2H), 7.36 (d, J=7.5 Hz, 1H), 6.85 (d, J=7.5 Hz, 1H), 3.69 (s, 2H), 2.70-2.55 (m, 4H), 1.91-1.75 (m, 2H) ppm. HR-MS(ESI): Calculated for C₂₃H₁₉F₂N₆OS [M+H]⁺: 465.1309. found 465.1298.

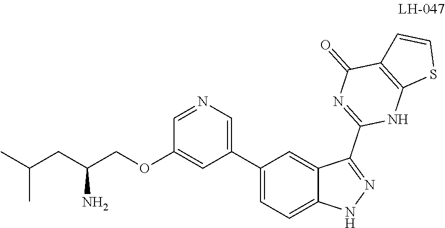 [SMILES: CC(C)C[C@H](N)COc1cncc(-c2ccc3[nH]nc(-c4nc(=O)c5ccsc5[nH]4)c3c2)c1]

¹H NMR (300 MHz, DMSO-d₆) δ 12.24 (s, 1H), 10.12 (s, 1H), 8.77 (t, J=1.0 Hz, 1H), 8.60 (d, J=1.3 Hz, 1H), 8.18 (d, J=1.3 Hz, 1H), 7.60 (dt, J=15.1, 1.4 Hz, 3H), 7.36 (d, J=7.6 Hz, 1H), 6.85 (d, J=7.5 Hz, 1H), 4.31 (dd, J=12.4, 7.0 Hz, 1H), 4.09 (dd, J=12.4, 7.0 Hz, 1H), 3.13-2.92 (m, 1H), 1.75-1.41 (m, 3H), 1.52 (s, 2H), 0.90 (dd, J=15.0, 6.5 Hz, 6H) ppm. HR-MS(ESI): Calculated for C₂₄H₂₅N₆O₂S [M+H]⁺: 461.1760. found 461.1756.

### Example 6: Synthesis of LH-048

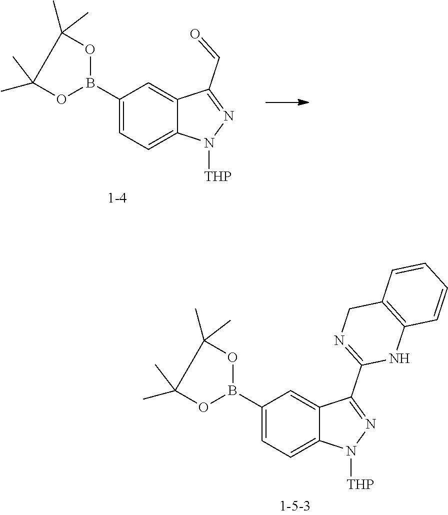

Intermediate 1-4 in route 1 (0.1 mmol) was added to a single-necked flask and dissolved with 10 mL of dimethylacetamide. o-Aminobenzylamine (0.15 mmol), p-toluenesulfonic acid hydrate (0.15 mmol), and sodium bisulfite (0.15 mmol) were then added, and the mixture was reacted at 120° C. for 6-8 h. After the TLC reaction was completed, the reaction mixture was extracted with ethyl acetate and saturated brine three times. The organic phases were combined, dried, concentrated, and purified by column chromatography to give intermediate 1-5-3. ¹H NMR (300 MHz, DMSO-d₆) δ 12.24 (s, 1H). 7.99 (d, J=1.5 Hz, 1H), 7.53 (d, J=7.5 Hz, 1H), 7.34 (dd, J=7.5, 1.5 Hz, 1H), 7.15-7.10 (m, 1H), 7.04 (td, J=7.4, 2.0 Hz, 1H), 6.89 (td, J=7.5, 2.1 Hz, 1H), 6.41 (dd, J=7.4, 2.1 Hz, 1H), 6.26 (t, J=6.9 Hz, 1H), 4.94 (d, J=1.1 Hz, 2H), 3.90 (dt, J=11.4, 7.0 Hz, 1H), 3.77 (dt, J=11.5, 7.0 Hz, 1H), 2.52-2.32 (m, 1H), 2.12-1.91 (m, 2H), 1.86-1.48 (m, 3H), 1.41 (d, J=14.9 Hz, 12H) ppm. HR-MS(ESI): Calculated for C₂₆H₃₂BN₄O₃ [M+H]⁺: 459.2567. found 459.2558. The yield was 78%.

The other steps were the same as those in Example 1 to give compound LH-048.

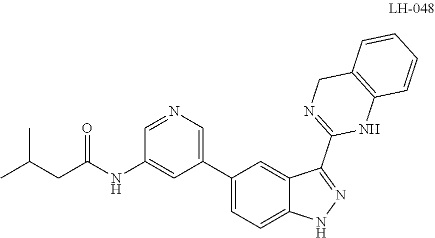 [SMILES: CC(C)CC(=O)Nc1cncc(-c2ccc3[nH]nc(C4=NCc5ccccc5N4)c3c2)c1]

¹H NMR (300 MHz, DMSO-d₆) § 12.36 (s, 1H), 10.28 (s, 1H), 9.80 (s, 1H), 9.51 (d, J=1.3 Hz, 1H), 8.67 (d, J=1.3 Hz, 1H), 8.47 (d, J=1.5 Hz, 1H), 7.99 (t, J=1.3 Hz, 1H), 7.62-7.47 (m, 2H), 7.15-7.11 (m, 1H), 7.04 (td, J=7.5, 2.1 Hz, 1H), 6.90 (td, J=7.5, 2.1 Hz, 1H), 6.41 (dd, J=7.5, 2.1 Hz, 1H), 2.60 (d, J=1.0 Hz, 2H), 2.29 (dd, J=13.5, 6.8 Hz, 1H), 2.10 (d, J=6.9 Hz, 2H), 0.96 (d, J=6.7 Hz, 6H) ppm. HR-MS(ESI): Calculated for C₂₅H₂₅N₆O [M+H]⁺: 425.2090. found 425.2088.

The following compounds were prepared using a procedure similar to that in Example 6:

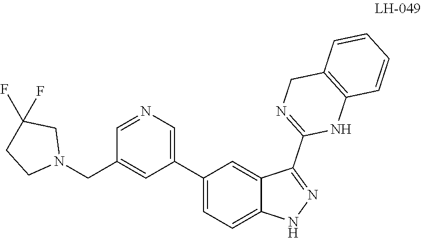 [SMILES: FC1(F)CCN(Cc2cncc(-c3ccc4[nH]nc(C5=NCc6ccccc6N5)c4c3)c2)C1]

¹H NMR (300 MHz, DMSO-d₆) § 12.11 (s, 1H), 10.22 (s, 1H), 8.82 (d, J=1.3 Hz, 1H), 8.71 (d, J=1.3 Hz, 1H), 8.48 (d, J=1.4 Hz, 1H), 8.08 (t, J=1.3 Hz, 1H), 7.60-7.48 (m, 2H), 7.15-7.05 (m, 1H), 7.04 (td, J=7.5, 2.0 Hz, 1H), 6.90 (td, J=7.4, 2.0 Hz, 1H), 6.41 (dd, J=7.5, 2.1 Hz, 1H), 3.69 (s, 2H), 2.70-2.55 (m, 2H), 2.60 (s, 3H), 1.91-1.75 (m, 2H) ppm. HR-MS(ESI): Calculated for C₂₅H₂₃F₂N₆ [M+H]⁺: 445.1952. found 445.1949.

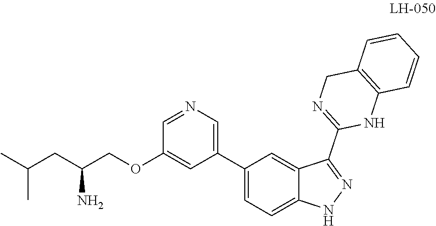 [SMILES: CC(C)C[C@H](N)COc1cncc(-c2ccc3[nH]nc(C4=NCc5ccccc5N4)c3c2)c1]

¹H NMR (300 MHz, DMSO-d₆) δ 14.21 (s, 1H), 12.31 (s, 1H), 8.56-8.42 (m, 2H), 8.15 (d, J=1.3 Hz, 1H), 7.57 (dd, J=13.9, 1.2 Hz, 3H), 7.15 (ddt, J=7.4, 2.1, 1.1 Hz, 1H), 7.04 (td, J=7.5, 2.1 Hz, 1H), 6.90 (td, J=7.4, 2.0 Hz, 1H), 6.41 (dd, J=7.5, 2.1 Hz, 1H), 4.95 (d, J=1.2 Hz, 2H), 4.33 (dd, J=12.4, 7.0 Hz, 1H), 4.08 (dd, J=12.4, 7.0 Hz, 1H), 3.13-3.07 (m, 1H), 1.74-1.39 (m, 3H), 1.56 (s, 2H), 0.90 (dd, J=15.0, 6.4 Hz, 6H) ppm. HR-MS(ESI): Calculated for C₂₆H₂₉N₆O [M+H]⁺: 441.2403. found 441.2395.

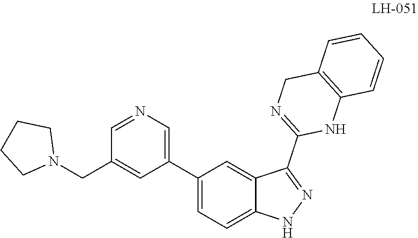 [SMILES: c1ccc2c(c1)CN=C(c1n[nH]c3ccc(-c4cncc(CN5CCCC5)c4)cc13)N2]

¹H NMR (300 MHz, DMSO-d₆) δ 12.32 (s, 1H), 10.21 (s, 1H), 8.82 (d, J=1.2 Hz, 1H), 8.69 (d, J=1.3 Hz, 1H), 8.49 (t, J=1.0 Hz, 1H), 8.10 (t, J=1.3 Hz, 1H), 7.55 (s, 1H), 7.15 (ddt, J=7.4, 2.1, 1.0 Hz, 1H), 7.04 (td, J=7.5, 2.1 Hz, 1H), 6.90 (td, J=7.4, 2.0 Hz, 1H), 6.41 (dd, J=7.4, 2.1 Hz, 1H), 3.69 (s, 2H), 2.68-2.51 (m, 3H), 2.60 (s, 4H), 1.91-1.74 (m, 4H) ppm. HR-MS(ESI): Calculated for C₂₅H₂₅N₆ [M+H]⁺: 409.2141. found 409.2098.

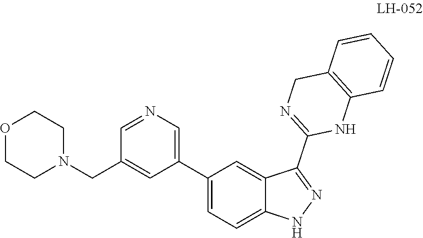 [SMILES: c1ccc2c(c1)CN=C(c1n[nH]c3ccc(-c4cncc(CN5CCOCC5)c4)cc13)N2]

¹H NMR (300 MHz, DMSO-d₆) δ 12.30 (s, 1H), 10.29 (s, 1H), 8.82 (d, J=1.3 Hz, 1H), 8.70 (d, J=1.3 Hz, 1H), 8.49 (d, J=1.9 Hz, 1H), 8.09 (t, J=1.3 Hz, 1H), 7.60-7.48 (m, 2H), 7.15 (ddt, J=7.5, 2.1, 1.0 Hz, 1H), 7.04 (td, J=7.5, 2.1 Hz, 1H), 6.90 (td, J=7.4, 2.0 Hz, 1H), 6.41 (dd, J=7.5, 2.1 Hz, 1H), 3.69 (s, 2H), 3.58 (t, J=4.7 Hz, 4H), 2.60 (d, J=1.0 Hz, 2H), 2.39 (t, J=4.7 Hz, 4H) ppm. HR-MS(ESI): Calculated for C₂₅H₂₅N₆O [M+H]⁺: 425.2090. found 425.2081.

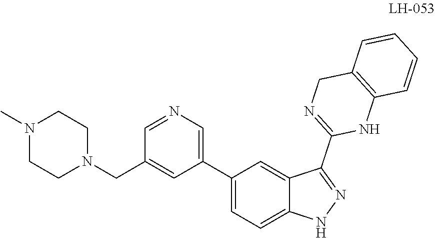 [SMILES: CN1CCN(Cc2cncc(-c3ccc4[nH]nc(C5=NCc6ccccc6N5)c4c3)c2)CC1]

¹H NMR (300 MHz, DMSO-d₆) δ 12.33 (s, 1H), 10.21 (s, 1H), 8.82 (d, J=1.3 Hz, 1H), 8.69 (d, J=1.3 Hz, 1H), 8.49 (t, J=1.0 Hz, 1H), 8.10 (t, J=1.3 Hz, 1H), 7.55 (s, 2H), 7.15 (ddt, J=7.5, 2.2, 1.0 Hz, 1H), 7.04 (td, J=7.5, 2.0 Hz, 1H), 6.90 (td, J=7.5, 2.1 Hz, 1H), 6.41 (dd, J=7.5, 2.1 Hz, 1H), 3.69 (s, 2H), 2.60 (d, J=1.0 Hz, 2H), 2.48 (t, J=5.1 Hz, 4H), 2.34 (t, J=5.2 Hz, 4H), 2.24 (s, 3H) ppm. HR-MS(ESI): Calculated for C₂₆H₂₈N₇ [M+H]⁺: 438.2406. found 438.2395.

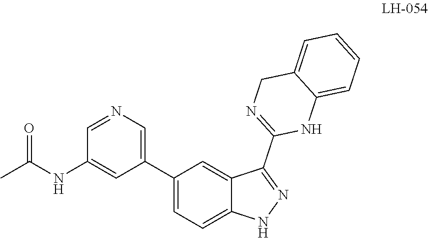 [SMILES: CC(=O)Nc1cncc(-c2ccc3[nH]nc(C4=NCc5ccccc5N4)c3c2)c1]

¹H NMR (300 MHz, DMSO-d₆) § 13.26 (s, 1H), 12.22 (s, 1H), 9.80 (s, 1H), 9.58 (d, J=1.3 Hz, 1H), 8.67 (d, J=1.3 Hz, 1H), 8.47 (d, J=1.5 Hz, 1H), 7.91 (t, J=1.2 Hz, 1H), 7.61-7.47 (m, 2H), 7.15 (ddt, J=7.4, 2.1, 1.0 Hz, 1H), 7.04 (td, J=7.5, 2.1 Hz, 1H), 6.90 (td, J=7.4, 2.0 Hz, 1H), 6.41 (dd, J=7.5, 2.1 Hz, 1H), 2.60 (d, J=1.0 Hz, 2H), 2.20 (s, 3H) ppm. HR-MS(ESI):

Calculated for C₂₂H₁₉N₆O [M+H]⁺: 383.1620. found 383.1598.

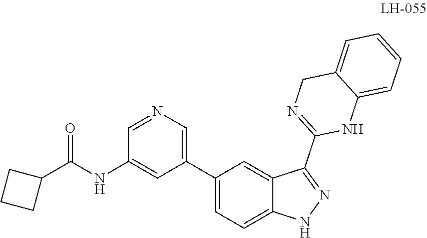 [SMILES: O=C(Nc1cncc(-c2ccc3[nH]nc(C4=NCc5ccccc5N4)c3c2)c1)C1CCC1]

¹H NMR (300 MHz, DMSO-d₆) δ 13.26 (s, 1H), 12.22 (s, 1H), 9.80 (s, 1H), 9.61 (d, J=1.3 Hz, 1H), 8.67 (d, J=1.3 Hz, 1H), 8.47 (d, J=1.4 Hz, 1H), 7.90 (t, J=1.3 Hz, 1H), 7.61-7.47 (m, 2H), 7.15 (ddt, J=7.4, 2.1, 1.0 Hz, 1H), 7.04 (td, J=7.5, 2.1 Hz, 1H), 6.90 (td, J=7.4, 2.0 Hz, 1H), 6.41 (dd, J=7.5, 2.1 Hz, 1H), 3.21 (d, J=6.9 Hz, 1H), 2.60 (d, J=1.0 Hz, 2H), 2.48 (dt, J=12.4, 6.8 Hz, 2H), 2.17-1.82 (m, 4H) ppm. HR-MS(ESI): Calculated for C₂₅H₂₃N₆O [M+H]⁺: 423.1933. found 423.1912.

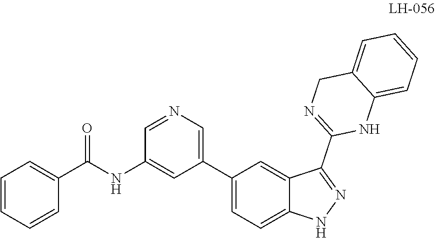 [SMILES: O=C(Nc1cncc(-c2ccc3[nH]nc(C4=NCc5ccccc5N4)c3c2)c1)c1ccccc1]

¹H NMR (300 MHz, DMSO-d₆) δ 14.26 (s, 1H), 12.22 (s, 1H), 9.50 (d, J=1.2 Hz, 1H), 8.68 (d, J=1.2 Hz, 1H), 8.49 (d, J=1.4 Hz, 1H), 8.04 (s, 1H), 8.00-7.88 (m, 3H), 7.62-7.47 (m, 4H), 7.31 (ddt, J=9.6, 7.2, 2.0 Hz, 1H), 7.15 (ddt, J=7.4, 2.2, 1.0 Hz, 1H), 7.04 (td, J=7.5, 2.1 Hz, 1H), 6.90 (td, J=7.5, 2.1 Hz, 1H), 6.41 (dd, J=7.4, 2.1 Hz, 1H), 2.60 (d, J=1.0 Hz, 2H) ppm. HR-MS(ESI): Calculated for C₂₇H₂₁N₆O [M+H]⁺: 445.1777. found 445.1765.

### Example 7: Synthesis of LH-057

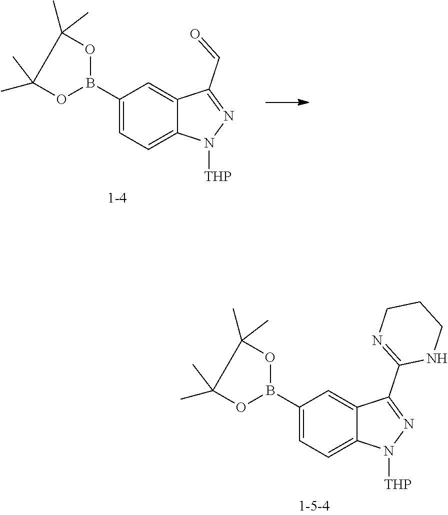

Intermediate 1-4 in route 1 (0.1 mmol) was added to a single-necked flask and dissolved with 5 mL of dimethylacetamide. Propanediamine (0.15 mmol), p-toluenesulfonic acid hydrate (0.15 mmol), and sodium bisulfite (0.15 mmol) were then added, and the mixture was reacted at 120° C. for 6-8 h. After the TLC reaction was completed, the reaction mixture was extracted with ethyl acetate and saturated brine for three times. The organic phases were combined, dried, concentrated, and purified by column chromatography to give intermediate 1-5-4. ¹H NMR (300 MHz, DMSO-d₆) δ 12.22 (s, 1H), 7.86 (d, J=1.5 Hz, 1H), 7.47-7.33 (m, 2H), 7.25 (dd, J=7.5, 1.6 Hz, 1H), 6.22 (t, J=6.8 Hz, 1H), 3.98-3.83 (m, 3H), 3.77 (dt, J=11.4, 7.0 Hz, 1H), 3.62 (ddd, J=11.9, 4.4, 1.7 Hz, 2H), 2.52-2.32 (m, 1H), 2.12-1.91 (m, 2H), 1.86-1.52 (m, 3H), 1.40 (d, J=15.1 Hz, 13H), 1.23-1.08 (m, 1H) ppm. HR-MS(ESI): Calculated for C₂₂H₃₂BN₄O₃ [M+H]⁺: 411.2567. found 411.2566. The yield was 78%.

The other steps were the same as those in Example 1 to give compound LH-057.

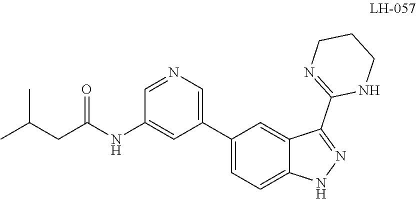 [SMILES: CC(C)CC(=O)Nc1cncc(-c2ccc3[nH]nc(C4=NCCCN4)c3c2)c1]

¹H NMR (300 MHz, DMSO-d₆) δ 14.32 (s, 1H), 9.80 (s, 1H), 9.58 (d, J=1.3 Hz, 1H), 8.65 (d, J=1.3 Hz, 1H), 8.38 (d, J=1.3 Hz, 1H), 7.92 (t, J=1.3 Hz, 1H), 7.60-7.43 (m, 3H), 3.52 (dt, J=9.2, 5.5 Hz, 4H), 2.30-2.10 (m, 2H), 2.09 (d, J=1.1 Hz, 1H), 1.60 (d, J=5.6 Hz, 2H), 0.97 (d, J=6.6 Hz, 6H) ppm. HR-MS(ESI): Calculated for C₂₁H₂₅N₆O [M+H]⁺: 377.2090. found 377.1998.

The following compounds were prepared using a procedure similar to that in Example 7:

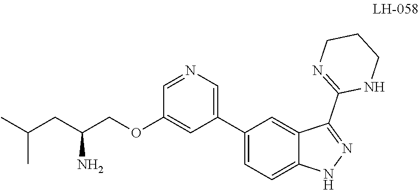 [SMILES: CC(C)C[C@H](N)COc1cncc(-c2ccc3[nH]nc(C4=NCCCN4)c3c2)c1]

¹H NMR (300 MHz, DMSO-d₆) δ 14.35 (s, 1H), 8.56 (d, J=1.3 Hz, 1H), 8.16 (d, J=1.3 Hz, 1H), 7.61 (t, J=1.3 Hz, 1H), 7.53 (d, J=1.1 Hz, 2H), 7.44 (s, 1H), 4.32 (dd, J=12.4, 7.0 Hz, 1H), 4.08 (dd, J=12.4, 7.0 Hz, 1H), 3.92 (td, J=12.2, 3.1 Hz, 2H), 3.66 (ddd, J=11.9, 4.4, 1.7 Hz, 2H), 3.20-3.04 (m, 1H), 1.74-1.33 (m, 6H), 1.25-1.12 (m, 1H), 0.90 (dd, J=15.0, 6.4 Hz, 6H) ppm. HR-MS(ESI): Calculated for C₂₂H₂₉N₆O [M+H]⁺: 393.2403. found 393.2403.

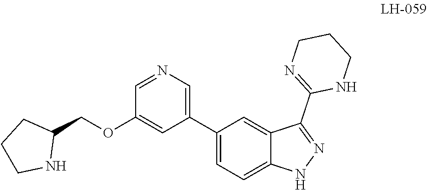

¹H NMR (300 MHz, DMSO-d₆) 14.35 (s, 1H), 8.56 (d, J=1.2 Hz, 1H), 8.38 (t, J=1.0 Hz, 1H), 8.17 (d, J=1.4 Hz, 1H), 7.57 (dd, J=15.2, 1.2 Hz, 3H), 7.45 (s, 1H), 3.93 (td, J=12.2, 3.1 Hz, 2H), 3.63 (ddd, J=11.9, 4.4, 1.7 Hz, 2H), 3.21 (d, J=6.9 Hz, 1H), 3.03-2.87 (m, 1H), 2.78 (dt, J=9.6, 7.0 Hz, 1H), 1.89-1.18 (m, 7H) ppm. HR-MS(ESI): Calculated for C₂₁H₂₃N₇O [M+H]⁺: 389.1964. found 389.1971.

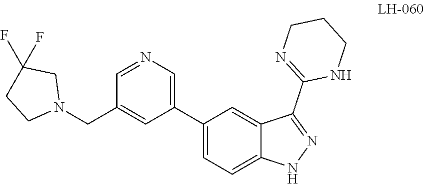 [SMILES: FC1(F)CCN(Cc2cncc(-c3ccc4[nH]nc(C5=NCCCN5)c4c3)c2)C1]

¹H NMR (300 MHz, DMSO-d₆) δ 14.36 (s, 1H), 8.79 (d, J=1.3 Hz, 1H), 8.71 (d, J=1.4 Hz, 1H), 8.37 (d, J=1.3 Hz, 1H), 8.07 (t, J=1.3 Hz, 1H), 7.59-7.42 (m, 3H), 3.69 (s, 2H), 3.52 (dt, J=9.2, 5.5 Hz, 4H), 2.70-2.55 (m, 4H), 1.91 (tt, J=15.9, 6.3 Hz, 2H), 1.60-1.55 (m, 2H) ppm. HR-MS(ESI): Calculated for C₂₁H₂₃F₂N₆ [M+H]⁺: 397.1952. found 397.1946.

### Example 8: In Vitro Inhibitory Activity of Some Compounds of the Present Invention Against the CLK Family and DYEK1A Proteins

### I. Experimental Procedures

### 1. Preparation of 1× Kinase Reaction Buffer

| | Stock solution | | | | Final | |
|---|---|---|---|---|---|---|
| Name | concentration | | Volume | | concentration | |
| Tris | 1M | (25X) | 240 | μL | 40 | mM |
| MgCl₂ | 1M | (50X) | 120 | μL | 20 | mM |
| BSA | 7.5% | (75X) | 80 | μL | 0.1% | |
| DTT | 1M | (500X) | 3 | μL | 0.5 | mM |

| | | |
|---|---|---|
| ddH₂O | 5557 | μL |

### 2. Enzyme Activity Assay

### (1) Preparation of 2× Kinase:

| | Stock solution | Final | |
|---|---|---|---|
| Name | concentration | concentration | |
| DYRK1A | 200 ng/μL | 0.2 | ng/μL |
| CLK1 | 100 ng/μL | 0.5 | ng/μL |
| CLK2 | 100 ng/μL | 0.5 | ng/μL |
| CLK3 | 100 ng/μL | 1 | ng/μL |
| CLK4 | 100 ng/μL | 0.5 | ng/μL |

### (2) Preparation of 4× Substrate Mixture:

| | |
|---|---|
| ATP | Substrate |

| | Final | Stock solution | | Final |
|---|---|---|---|---|
| Name | concentration | concentration | | concentration |
| DYRKIA | 30 μM | 5 | mg/mL | 0.1 mg/mL |
| CLK1 | 50 μM | 10 | mg/mL | 0.1 mg/mL |
| CLK2 | 50 μM | 10 | mg/mL | 0.1 mg/mL |
| CLK3 | 30 μM | 5 | mg/mL | 0.1 mg/mL |
| CLK4 | 50 μM | 10 | mg/mL | 0.1 mg/mL |

\textcircled{1} The positive drug was diluted in a 4-fold gradient with DMSO in a dilution plate, with the final initial concentrations of the compound being 1 & 0.02 & 0.1 μM.

\textcircled{2} The compound was diluted 50-fold into the 1× kinase reaction buffer and oscillated on an oscillator for 20 min.

\textcircled{3} The 2× kinase was prepared using the 1× enzyme reaction buffer.

\textcircled{4} 2 μL of the kinase was added per well to the reaction plate.

\textcircled{5} 1 μL of the compound diluted in the buffer was added to each well. The plate was sealed with a plate-sealing film, centrifuged at 1000 rpm for 60 s, and incubated at 25° C. for 10 min.

\textcircled{6} 4× ATP & sub mixture was prepared using the 1× enzyme reaction buffer, and 1 μL of the 4× ATP & sub mixture was added to the reaction plate.

\textcircled{7} The plate was sealed with a plate-sealing film, centrifuged at 1000 rpm for 60 s, and incubated at 25° C. for 60 min.

\textcircled{8} 4 μL of ADP-Glo was transferred to the 384 reaction plate, and the plate was centrifuged at 1000 rpm for 1 min and incubated at 25° C. for 40 min.

\textcircled{9} 8 μL of Detection solution was transferred to the 384 reaction plate, and the plate was centrifuged at 1000 rpm for 1 min and incubated at 25° C. for 40 min.

\textcircled{10} The RLU (relative luminescence unit) signal was read using a BMG multifunctional microplate reader, and the signal intensity was used to characterize the degree of the kinase activity.

### 3. Data Analysis

(1) the ratio of each well was calculated;
(2) the inhibition rate was calculated as follows:

compound inhibition rate (% inh)=100%−(compound−positive control)/(negative control−positive control)×100%
(3) the IC₅₀ was calculated, and the inhibition curves of the compounds were plotted:
the following nonlinear fitting formula was used to obtain the IC₅₀ (half maximal inhibitory concentration) of the compounds: Graphpad 7.0 software was used for data analysis.

$$Y = Bottom + ( Top - Bottom ) / ( 1 + 10 \hat{} ( ( Log {IC}_{50} - X ) \times Hill Slope ) ) ;$$

X: log value of the concentration of the compound, Y: inhibition rate (% inhibition).

### II. Experimental Results

TABLE 1
Enzymatic activity (IC₅₀) of some of
compounds in the present invention

| ID | CLK1 | CLK3 | CLK4 | CLK2 | DYRK1A |
|---|---|---|---|---|---|
| LH-001 | A | A | C | A | B |
| LH-003 | A | A | D | A | C |
| LH-004 | A | B | C | B | B |
| LH-005 | C | C | B | B | B |
| LH-006 | B | C | B | B | B |
| LH-009 | A | A | B | A | A |
| LH-010 | B | B | D | A | B |
| LH-011 | C | C | D | A | C |
| LH-015 | B | B | A | A | A |
| LH-016 | A | B | B | A | B |
| LH-017 | A | D | C | A | A |
| LH-018 | C | B | A | A | A |
| LH-019 | A | D | C | A | B |
| LH-020 | A | C | B | A | A |
| LH-037 | A | A | B | B | A |
| LH-039 | C | A | C | B | B |
| LH-040 | C | B | A | A | A |
| LH-041 | B | A | B | A | B |
| LH-048 | A | A | B | A | A |
| LH-049 | C | B | D | A | A |

Note:
A: <10 nM, B: 10-50 nM, C: 50-100 nM, D: >100 nM.

As can be seen from Table 1, the compounds described in the present invention show effective inhibitory activity against CLK2 and DYRK1A. Meanwhile, the compounds described in the present invention, such as LH-020, show excellent CLK2 inhibitory activity and CLK3 selectivity (IC₅₀ for CLK2, CLK3 and DYRK1A was 2 nM, 81 nM and 3 nM, respectively, and the selectivity index for CLK3 was 41.5), which provides a basis for LH-020 to exert its pharmacological activity and avoid possible side effects.

### Example 9: Pharmacodynamic (PD) Results of In Vivo of Representative Compound LH-020 of the Present Invention in Osteoarthritis Rats

### I. Experimental Procedures

1. 40 rats were evenly divided into 4 groups, namely, Control (normal rats injected with physiological saline into the joint cavity), Model (ACLT Model rats injected with physiological saline into the joint cavity), low-dose administration group (ACLT Model rats injected with LH020 (1.5 μg/kg) into the joint cavity), and high-dose administration group (ACLT Model rats injected with LH020 (6 μg/kg) into the joint cavity).

2. Knee osteoarthritis was induced by resection of anterior cruciate ligament of the rats (ACLT model).

3. The first week after surgery, physiological saline or LH-020 (1.5 μg/kg. 6 μg/kg) was injected into the joint cavity.

4. Week 5 after administration, cartilage was collected, and the expression of cartilage-associated proteases (MMP3, MMP13, ADAMTS5, IHH, etc.) was detected by RT-qPCR.

### II. Experimental Results

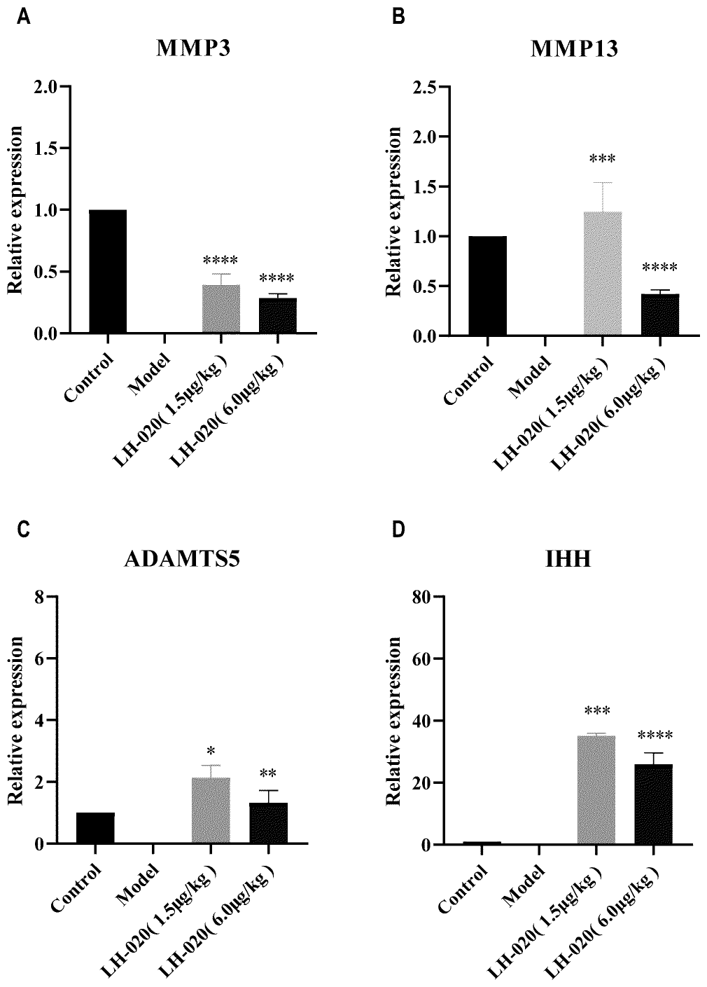

As can be seen in FIG. 1, as compared with the Control group (physiological saline was injected into the joint cavity), the expression of cartilage-degrading proteases MMP3, MMP13, ADAMTS5 and IHH was significantly increased in the arthritis model. In addition, as compared with the model group, the low-dose group and the high-dose group of LH-020 can significantly down-regulate the expression of MMP3, MMP13, ADAMTS5 and IHH, and the effect of the high-dose group was superior to that of the low-dose group, which shows that LH-020 has a significant chondroprotective effect and provides a basis for it to exert a pharmacological effect of treating osteoarthritis.

1. A 5-pyridine-1H-indazole compound, having a structure of formula I or II, wherein the compound further comprises an isomer thereof, a pharmaceutically acceptable salt thereof, or a mixture thereof: 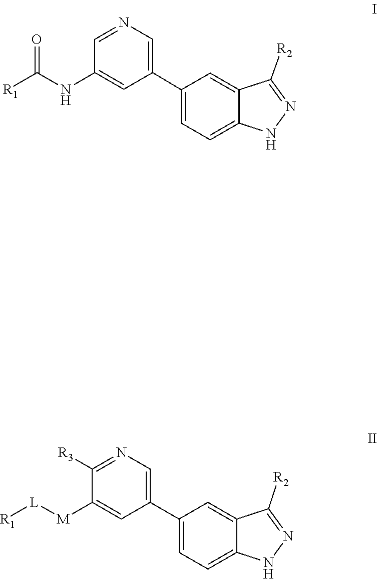
R₁ is selected from the following groups substituted with one or more of hydrogen, halogen, methoxy, trifluoromethyl, nitro, hydroxy, amino, azido, sulfonic acid group, or 3-6 membered ring: hydrogen, linear or branched C₁-C₁₀ alkyl, phenyl, a 4-6 membered heterocyclic ring, or a 4-6 membered ring;
L and M are selected from —CH₂—, —NH—, —O—, or a chemical bond;
R₂ is selected from the following groups: 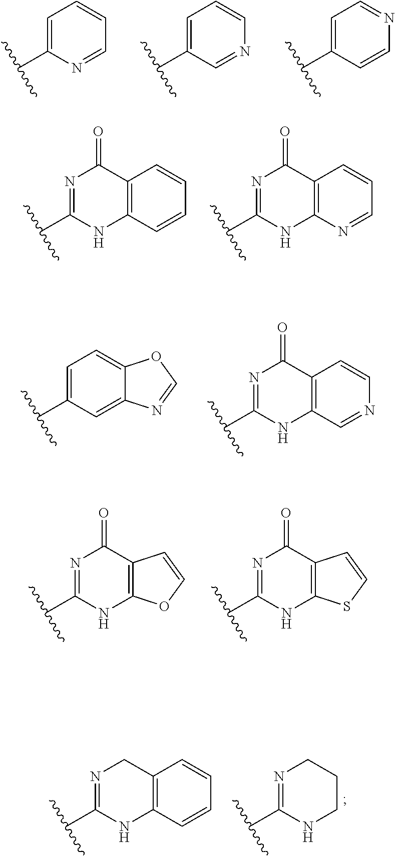
R₃ is selected from hydrogen or C₁-C₄ alkyl.

2. The compound according to claim 1, wherein in the structure:
R₁ is selected from isobutyl, cyclopropylmethyl, cyclopentyl, α-aminoisopentyl, 3,3-difluorotetrahydropyrrolyl, hydrogen, morpholinyl, methyl, tert-butyl, ethanesulfonyl, or hydroxy;
R₂ is selected from the following groups:

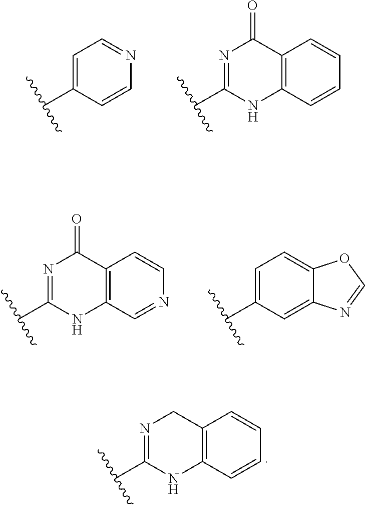 [SMILES: *c1ccncc1 ; *c1nc(=O)c2ccccc2[nH]1 ; *c1nc(=O)c2ccncc2[nH]1 ; *c1ccc2ocnc2c1 ; *C1=NCc2ccccc2N1]

3. The compound according to claim 1, wherein the compound is selected from any one of the following compounds:

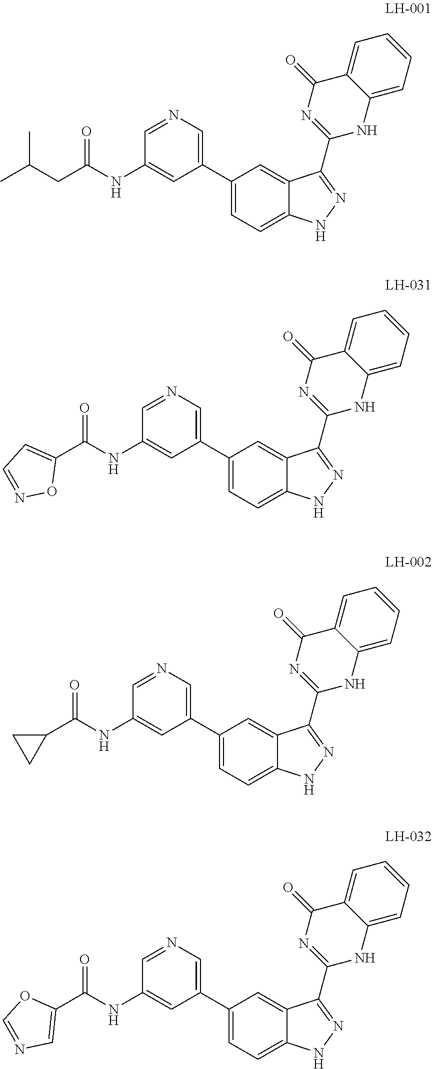 [SMILES: CC(C)CC(=O)Nc1cncc(-c2ccc3[nH]nc(-c4nc(=O)c5ccccc5[nH]4)c3c2)c1 ; O=C(Nc1cncc(-c2ccc3[nH]nc(-c4nc(=O)c5ccccc5[nH]4)c3c2)c1)c1ccno1 ; O=C(Nc1cncc(-c2ccc3[nH]nc(-c4nc(=O)c5ccccc5[nH]4)c3c2)c1)C1CC1 ; O=C(Nc1cncc(-c2ccc3[nH]nc(-c4nc(=O)c5ccccc5[nH]4)c3c2)c1)c1cnco1]

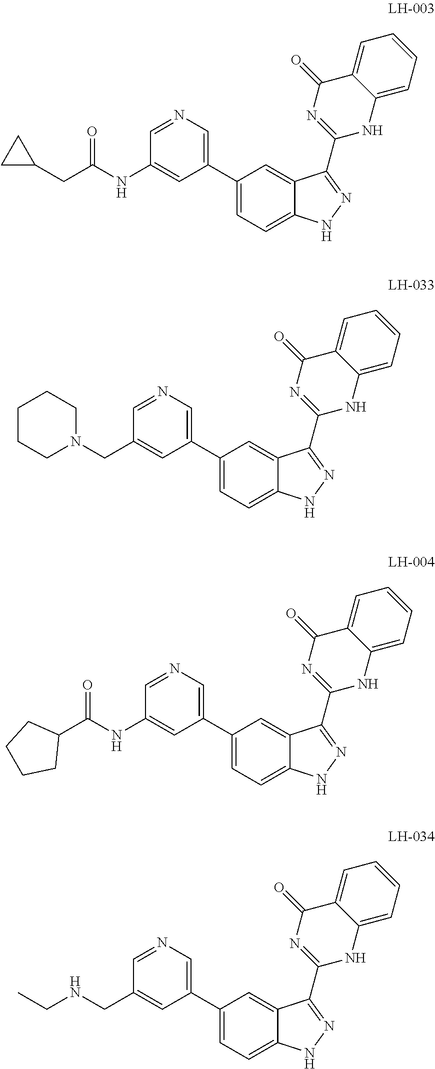 [SMILES: O=C(CC1CC1)Nc1cncc(-c2ccc3[nH]nc(-c4nc(=O)c5ccccc5[nH]4)c3c2)c1 ; O=c1nc(-c2n[nH]c3ccc(-c4cncc(CN5CCCCC5)c4)cc23)[nH]c2ccccc12 ; O=C(Nc1cncc(-c2ccc3[nH]nc(-c4nc(=O)c5ccccc5[nH]4)c3c2)c1)C1CCCC1 ; CCNCc1cncc(-c2ccc3[nH]nc(-c4nc(=O)c5ccccc5[nH]4)c3c2)c1]

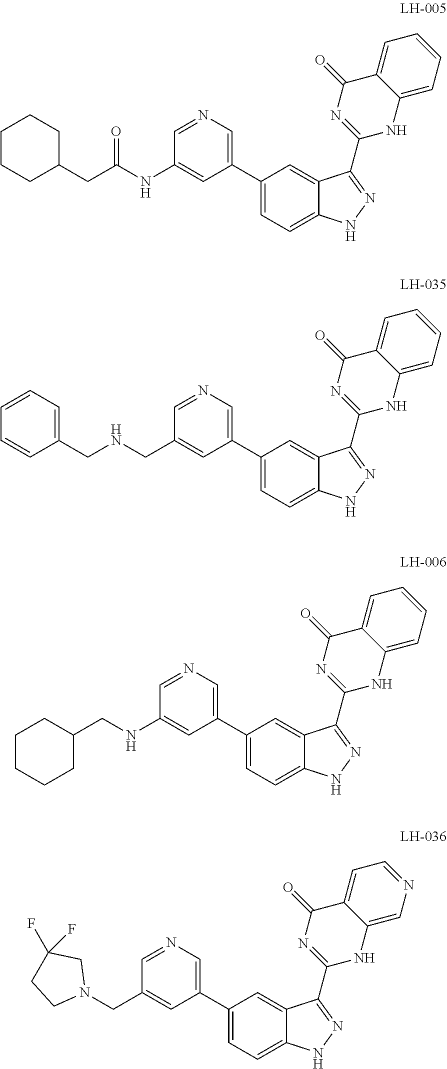 [SMILES: O=C(CC1CCCCC1)Nc1cncc(-c2ccc3[nH]nc(-c4nc(=O)c5ccccc5[nH]4)c3c2)c1 ; O=c1nc(-c2n[nH]c3ccc(-c4cncc(CNCc5ccccc5)c4)cc23)[nH]c2ccccc12 ; O=c1nc(-c2n[nH]c3ccc(-c4cncc(NCC5CCCCC5)c4)cc23)[nH]c2ccccc12 ; O=c1nc(-c2n[nH]c3ccc(-c4cncc(CN5CCC(F)(F)C5)c4)cc23)[nH]c2cnccc12]

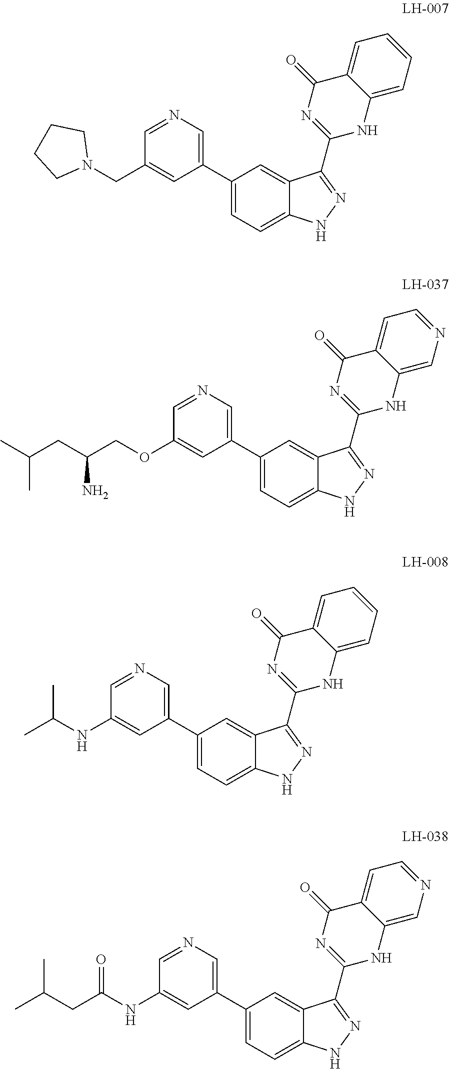 [SMILES: O=c1nc(-c2n[nH]c3ccc(-c4cncc(CN5CCCC5)c4)cc23)[nH]c2ccccc12 ; CC(C)C[C@H](N)COc1cncc(-c2ccc3[nH]nc(-c4nc(=O)c5ccncc5[nH]4)c3c2)c1 ; CC(C)Nc1cncc(-c2ccc3[nH]nc(-c4nc(=O)c5ccccc5[nH]4)c3c2)c1 ; CC(C)CC(=O)Nc1cncc(-c2ccc3[nH]nc(-c4nc(=O)c5ccncc5[nH]4)c3c2)c1]

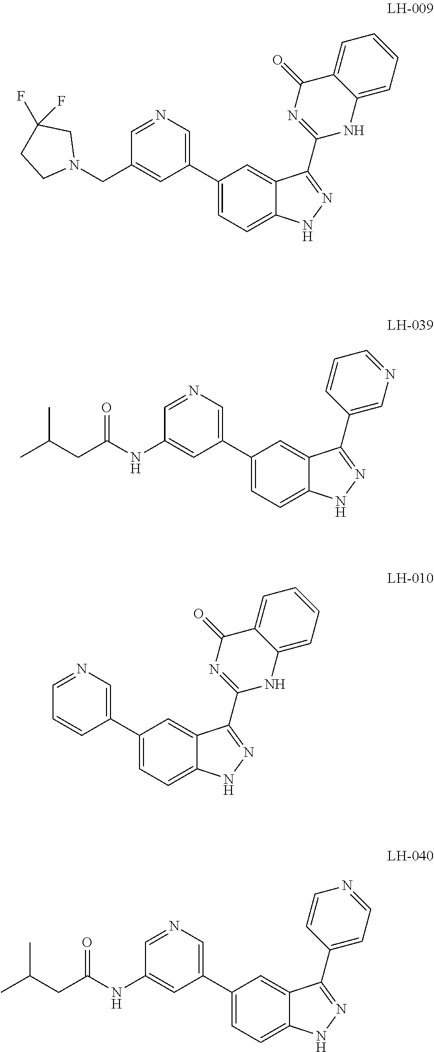 [SMILES: O=c1nc(-c2n[nH]c3ccc(-c4cncc(CN5CCC(F)(F)C5)c4)cc23)[nH]c2ccccc12 ; CC(C)CC(=O)Nc1cncc(-c2ccc3[nH]nc(-c4cccnc4)c3c2)c1 ; O=c1nc(-c2n[nH]c3ccc(-c4cccnc4)cc23)[nH]c2ccccc12 ; CC(C)CC(=O)Nc1cncc(-c2ccc3[nH]nc(-c4ccncc4)c3c2)c1]

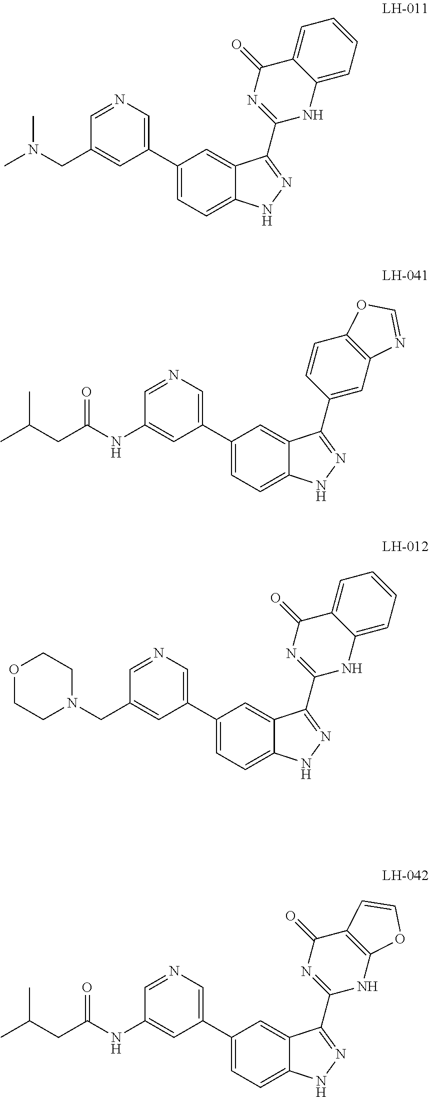 [SMILES: CN(C)Cc1cncc(-c2ccc3[nH]nc(-c4nc(=O)c5ccccc5[nH]4)c3c2)c1 ; CC(C)CC(=O)Nc1cncc(-c2ccc3[nH]nc(-c4ccc5ocnc5c4)c3c2)c1 ; O=c1nc(-c2n[nH]c3ccc(-c4cncc(CN5CCOCC5)c4)cc23)[nH]c2ccccc12 ; CC(C)CC(=O)Nc1cncc(-c2ccc3[nH]nc(-c4nc(=O)c5ccoc5[nH]4)c3c2)c1]

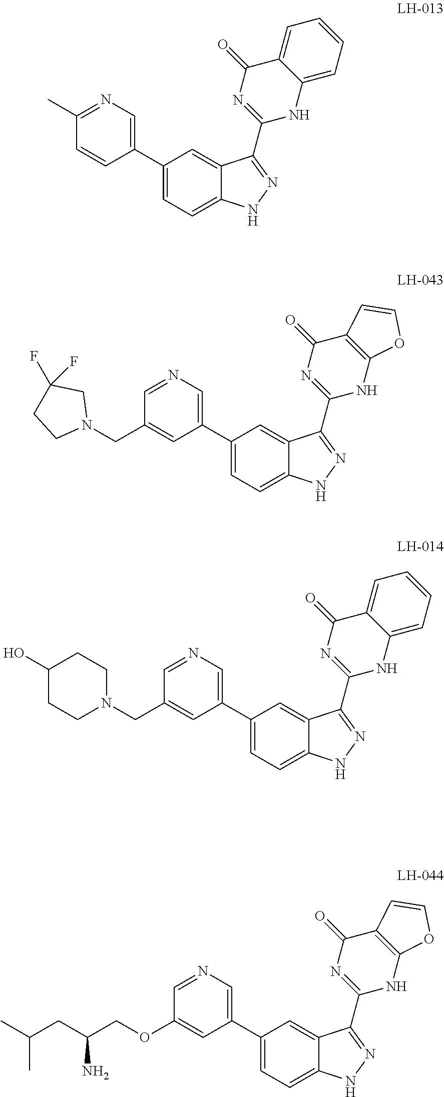 [SMILES: Cc1ccc(-c2ccc3[nH]nc(-c4nc(=O)c5ccccc5[nH]4)c3c2)cn1 ; O=c1nc(-c2n[nH]c3ccc(-c4cncc(CN5CCC(F)(F)C5)c4)cc23)[nH]c2occc12 ; O=c1nc(-c2n[nH]c3ccc(-c4cncc(CN5CCC(O)CC5)c4)cc23)[nH]c2ccccc12 ; CC(C)C[C@H](N)COc1cncc(-c2ccc3[nH]nc(-c4nc(=O)c5ccoc5[nH]4)c3c2)c1]

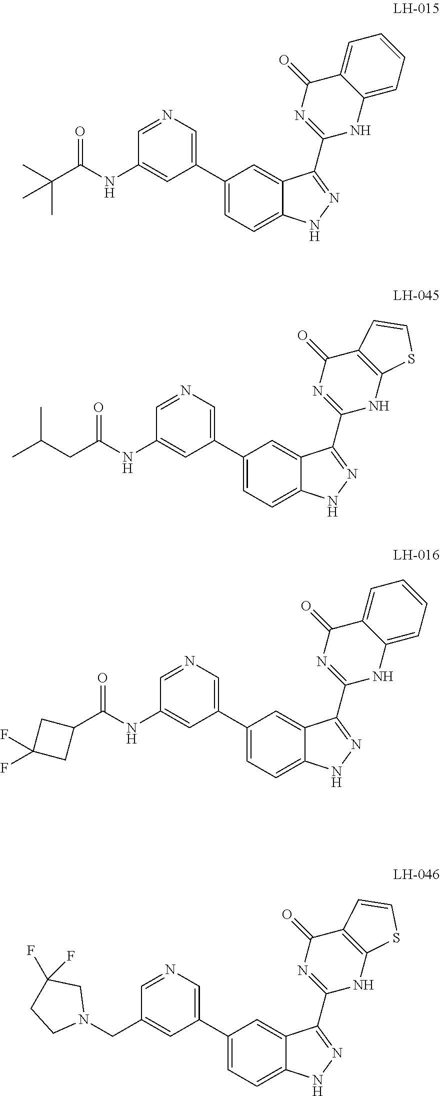 [SMILES: CC(C)(C)C(=O)Nc1cncc(-c2ccc3[nH]nc(-c4nc(=O)c5ccccc5[nH]4)c3c2)c1 ; CC(C)CC(=O)Nc1cncc(-c2ccc3[nH]nc(-c4nc(=O)c5ccsc5[nH]4)c3c2)c1 ; O=C(Nc1cncc(-c2ccc3[nH]nc(-c4nc(=O)c5ccccc5[nH]4)c3c2)c1)C1CC(F)(F)C1 ; O=c1nc(-c2n[nH]c3ccc(-c4cncc(CN5CCC(F)(F)C5)c4)cc23)[nH]c2sccc12]

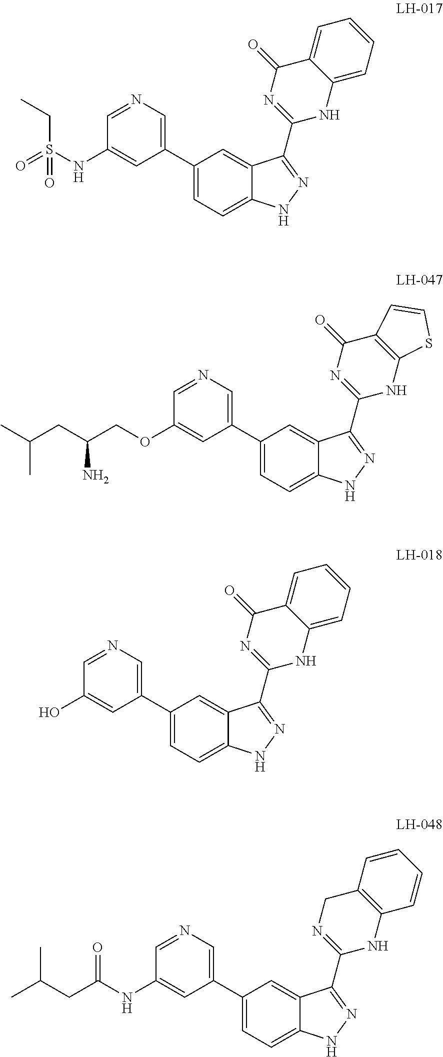 [SMILES: CCS(=O)(=O)Nc1cncc(-c2ccc3[nH]nc(-c4nc(=O)c5ccccc5[nH]4)c3c2)c1 ; CC(C)C[C@H](N)COc1cncc(-c2ccc3[nH]nc(-c4nc(=O)c5ccsc5[nH]4)c3c2)c1 ; O=c1nc(-c2n[nH]c3ccc(-c4cncc(O)c4)cc23)[nH]c2ccccc12 ; CC(C)CC(=O)Nc1cncc(-c2ccc3[nH]nc(C4=NCc5ccccc5N4)c3c2)c1]

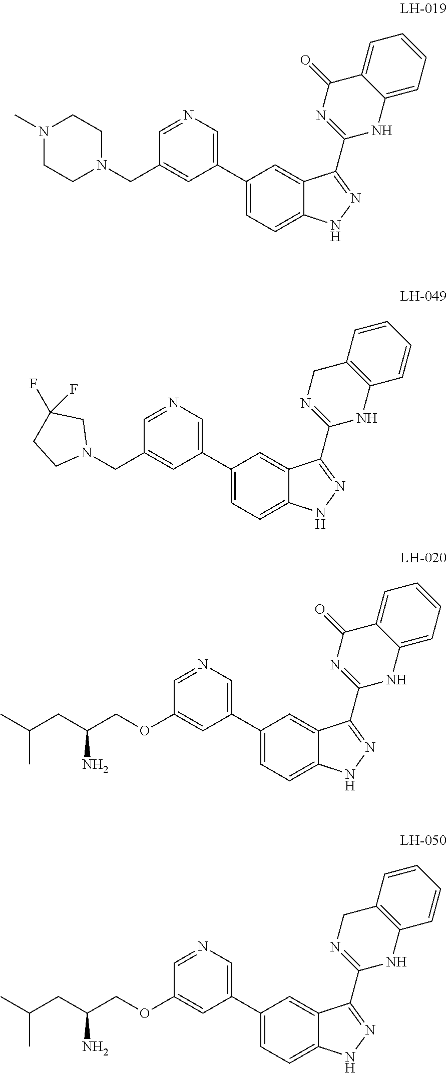 [SMILES: CN1CCN(Cc2cncc(-c3ccc4[nH]nc(-c5nc(=O)c6ccccc6[nH]5)c4c3)c2)CC1 ; FC1(F)CCN(Cc2cncc(-c3ccc4[nH]nc(C5=NCc6ccccc6N5)c4c3)c2)C1 ; CC(C)C[C@H](N)COc1cncc(-c2ccc3[nH]nc(-c4nc(=O)c5ccccc5[nH]4)c3c2)c1 ; CC(C)C[C@H](N)COc1cncc(-c2ccc3[nH]nc(C4=NCc5ccccc5N4)c3c2)c1]

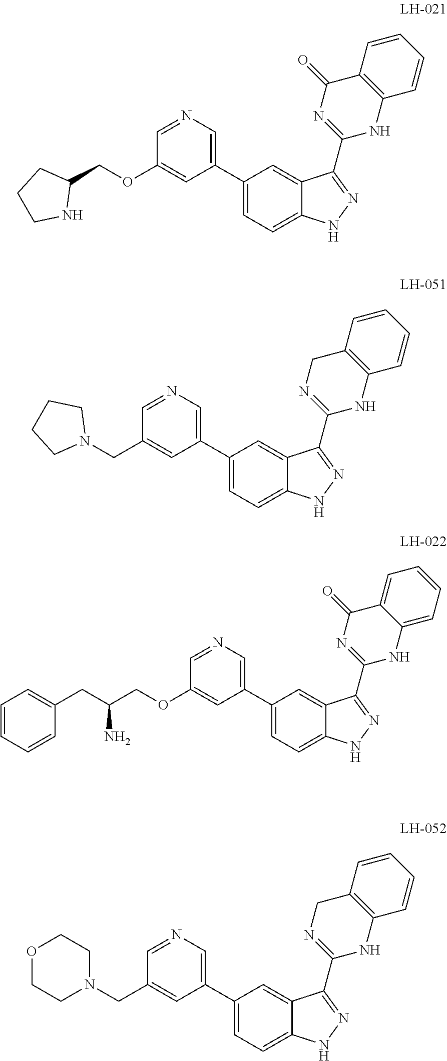

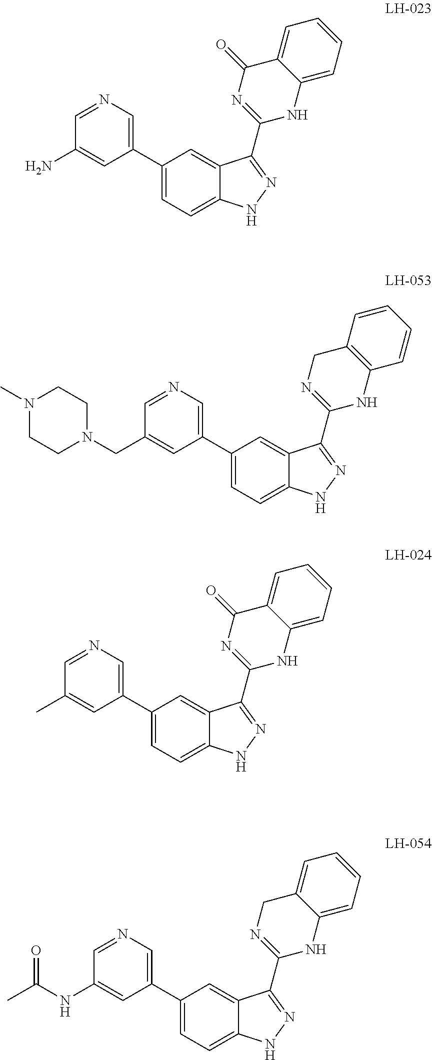 [SMILES: Nc1cncc(-c2ccc3[nH]nc(-c4nc(=O)c5ccccc5[nH]4)c3c2)c1 ; CN1CCN(Cc2cncc(-c3ccc4[nH]nc(C5=NCc6ccccc6N5)c4c3)c2)CC1 ; Cc1cncc(-c2ccc3[nH]nc(-c4nc(=O)c5ccccc5[nH]4)c3c2)c1 ; CC(=O)Nc1cncc(-c2ccc3[nH]nc(C4=NCc5ccccc5N4)c3c2)c1]

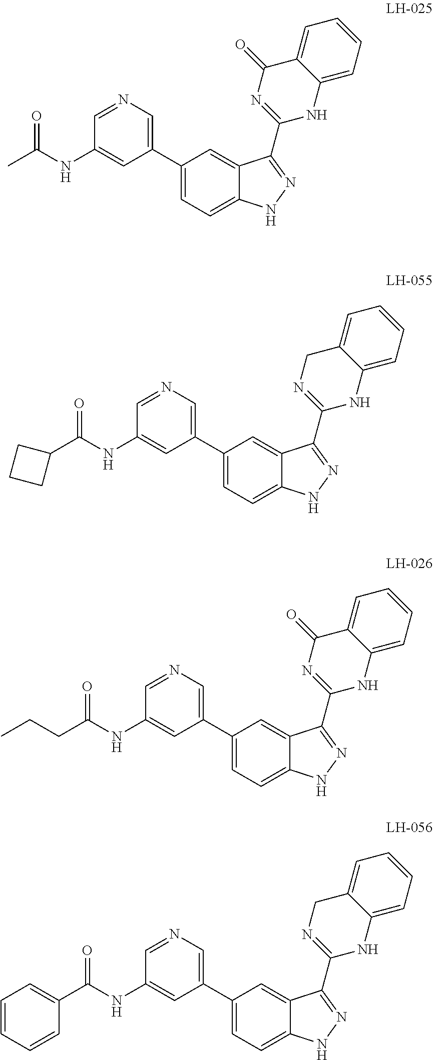 [SMILES: CC(=O)Nc1cncc(-c2ccc3[nH]nc(-c4nc(=O)c5ccccc5[nH]4)c3c2)c1 ; O=C(Nc1cncc(-c2ccc3[nH]nc(C4=NCc5ccccc5N4)c3c2)c1)C1CCC1 ; CCCC(=O)Nc1cncc(-c2ccc3[nH]nc(-c4nc(=O)c5ccccc5[nH]4)c3c2)c1 ; O=C(Nc1cncc(-c2ccc3[nH]nc(C4=NCc5ccccc5N4)c3c2)c1)c1ccccc1]

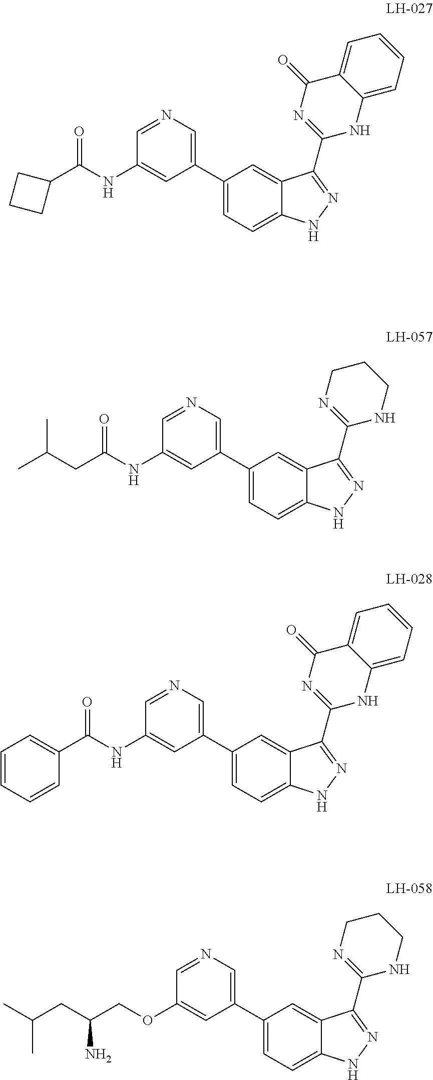 [SMILES: O=C(Nc1cncc(-c2ccc3[nH]nc(-c4nc(=O)c5ccccc5[nH]4)c3c2)c1)C1CCC1 ; CC(C)CC(=O)Nc1cncc(-c2ccc3[nH]nc(C4=NCCCN4)c3c2)c1 ; O=C(Nc1cncc(-c2ccc3[nH]nc(-c4nc(=O)c5ccccc5[nH]4)c3c2)c1)c1ccccc1 ; CC(C)C[C@H](N)COc1cncc(-c2ccc3[nH]nc(C4=NCCCN4)c3c2)c1]

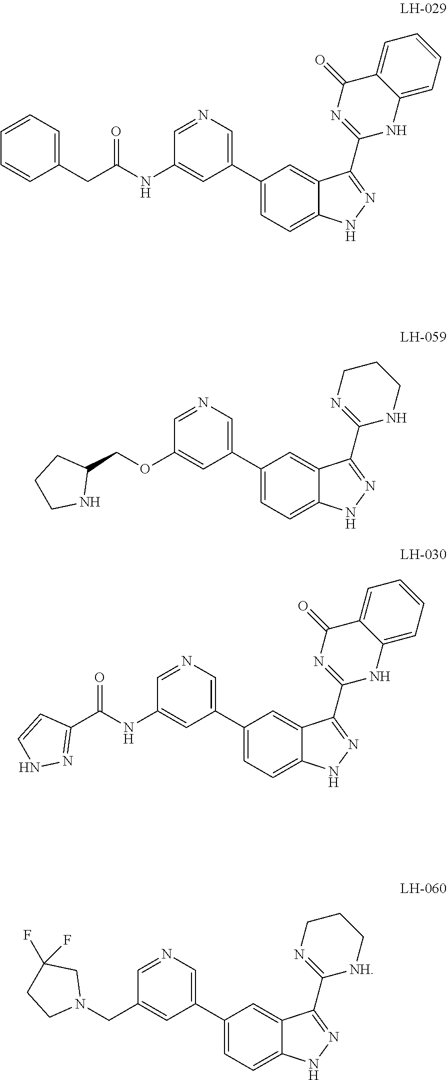

4. The compound according to claim 1, wherein the pharmaceutically acceptable salt is a salt formed by the compound and the following acids: hydrochloric acid, sulfuric acid, phosphoric acid, carbonic acid, nitric acid, hydrobromic acid, hydroiodic acid, maleic acid, fumaric acid, tartaric acid, citric acid, malic acid, methanesulfonic acid, p-toluenesulfonic acid, naphthalenesulfonic acid, succinic acid, acetic acid, mandelic acid, isobutyric acid, or malonic acid.

5. A pharmaceutical composition, wherein comprising the compound according to claim 1 and a pharmaceutically acceptable carrier.

6. Use of the pharmaceutical composition according to claim 5 in the preparation of an inhibitor drug for CLK2 protein.

7. Use of the pharmaceutical composition according to claim 5 in the preparation of an inhibitor drug for DYRK1A protein.

8. The use according to claim 6, wherein the drug is a drug for treating inflammation.

9. The use according to claim 8, wherein the inflammation is osteoarthritis, tendinopathy, or rheumatoid arthritis.

10. The use according to claim 9, wherein the compound has a chondroprotective effect.

11. The use according to claim 7, wherein the drug is a drug for treating inflammation.

12. The use according to claim 11, wherein the inflammation is osteoarthritis, tendinopathy, or rheumatoid arthritis.

13. The use according to claim 12, wherein the compound has a chondroprotective effect.
